(19) United States
(12) Patent Application Publication
(10) Pub. No.: US 2026/0053421 A1
(43) Pub. Date: Feb. 26, 2026

(54) POST TRAUMATIC STRESS DISORDER TREATMENT

(71) Applicant: GrayMatters Health Ltd., Haifa (IL)

(72) Inventors: Oded Shlomo KRAFT-OZ, Haifa (IL); Ran COHEN, Tel-Aviv (IL); Shai ATTIA, Shimshit (IL); Dmitri LAVRO, Kfar Vradim (IL); Adar SHANI, Pardes Hana (IL); Yaakov STERN, Sde Yaakov (IL)

(73) Assignee: GrayMatters Health Ltd., Haifa (IL)

(21) Appl. No.: 19/103,830

(22) PCT Filed: Aug. 16, 2023
(86) PCT No.: PCT/IL2023/050865

§ 371 Date: Feb. 14, 2025

Related U.S. Application Data
(60) Provisional application No. 63/398,248, filed on Aug. 16, 2022.

(51) Int. Cl. A61B 5/375; A61B 5/00; A61B 5/372

(52) U.S. Cl. CPC A61B 5/375; A61B 5/372; A61B 5/7405; A61B 5/742

(57) ABSTRACT

A method to treat a predetermined patient population for post traumatic stress disorder (PTSD), the method including:
selecting a population of patients diagnosed with PTSD;
delivering a NF treatment to the population;
achieving a reduction of at least 6 points in average in a CAPS-5 scale in the selected population, after a time period between 8 weeks and 12 weeks of the delivering.

### RELATED APPLICATION/S

This application claims the benefit of priority under 35 USC § 119(e) of U.S. Provisional Patent Application No. 63/398,248 filed Aug. 16, 2022, the contents of which are incorporated herein by reference in their entirety.

### FIELD AND BACKGROUND OF THE INVENTION

The present invention, in some embodiments thereof, relates to a stress disorder treatment and, more particularly, but not exclusively, to a post-traumatic stress disorder (PTSD) treatment.

### SUMMARY OF THE INVENTION

Some examples of some embodiments of the invention are listed below (an embodiment may include features from more than one example and/or fewer than all features of an example):

Example 1. A method to treat a predetermined patient population for post traumatic stress disorder (PTSD), the method comprising:
selecting a population of patients diagnosed with PTSD;
delivering a NF treatment to said population;
achieving a reduction of at least 6 points in average in a CAPS-5 scale in said selected population, after a time period between 8 weeks and 12 weeks of said delivering.

Example 2. A method according to example 1, wherein said delivering comprises training said selected population using said NF treatment to modulate activation of the Amygdala or activation of at least one related brain region or at least one biomarker thereof.

Example 3. A method according to example 2, wherein said training comprises training said selected population using said NF treatment to modulate said activation of the Amygdala or activation of said brain region or said at least one biomarker thereof, when exposed to at least one interface.

Example 4. A method according to example 3, wherein said delivered NF treatment comprises recording EEG signals from said selected population during said exposure to said at least one interface, extracting one or more signals which selectively indicate an activity level of said Amygdala or an activity level of said related brain region or said at least one biomarker thereof, from said recorded EEG signals, and providing feedback to said population regarding an activity of said Amygdala, or regarding an activity of said at least one related brain region or regarding said at least one biomarker thereof, based on the one or more extracted signals.

Example 5. A method according to any one of examples 1 to 3, wherein said delivered NF treatment is a functional magnetic resonance imaging (fMRI) neurofeedback treatment.

Example 6. A method according to any one of the previous examples, wherein said achieving comprises achieving a reduction of at least 6 points in average in said CAPS-5 scale, up to 3 months from said delivering.

Example 7. A method according to any one of the previous examples, wherein said selecting comprises selecting a population of patients diagnosed with chronic PTSD.

Example 8. A method to treat a predetermined patient population for post traumatic stress disorder (PTSD), the method comprising:
selecting a population of patients diagnosed with PTSD;
delivering a NF treatment to said population during a time period between 8 weeks and 12 weeks;
achieving a reduction of at least 6 points in average in a CAPS-5 scale in said selected population, after a time period up to 3 months from said delivering.

Example 9. A method according to example 8, wherein said delivering comprises training said selected population using said NF treatment to modulate activation of the Amygdala or activation of at least one related brain region or at least one biomarker thereof.

Example 10. A method according to example 9, wherein said training comprises training said selected population using said NF treatment to modulate said activation of the Amygdala or activation of said brain region or said at least one biomarker thereof, when exposed to at least one interface.

Example 11. A method according to example 10, wherein said delivered NF treatment comprises recording EEG signals from said selected population during said exposure to said at least one interface, extracting one or more signals which selectively indicate an activity level of said Amygdala or an activity level of said related brain region or said at least one biomarker thereof, from said recorded EEG signals, and providing feedback to said population regarding an activity of said Amygdala, or regarding an activity of said at least one related brain region or regarding said at least one biomarker thereof, based on the one or more extracted signals.

Example 12. A method according to any one of examples 8 to 10, wherein said delivered NF treatment is a functional magnetic resonance imaging (fMRI) neurofeedback treatment.

Example 13. A method according to any one of the previous examples, wherein said achieving comprises achieving a reduction of at least 6 points in average in said CAPS-5 scale, when said delivering is completed.

Example 14. A method according to any one of examples 8 to 13, wherein said selecting comprises selecting a population of patients diagnosed with chronic PTSD.

Example 15. A method to treat a predetermined patient population for PTSD, the method comprising:
selecting a population of female patients diagnosed with PTSD;
delivering a NF treatment to said population;
achieving a reduction of at least 6 points in average, in a CAPS-5 scale in said selected population, after a time period of at least 8 weeks of said delivering.

Example 16. A method according to example 15, wherein said delivering comprises training said selected population using said NF treatment to modulate activation of the Amygdala or activation of at least one related brain region or at least one biomarker thereof.

Example 17. A method according to example 16, wherein said training comprises training said selected population using said NF treatment to modulate said activation of the Amygdala or activation of said at least one related brain region or said at least one biomarker thereof, when exposed to at least one interface.

Example 18. A method according to example 17, wherein said delivered NF treatment comprises recording EEG signals from said selected population during said exposure to said at least one interface, extracting one or more signals which selectively indicate an activity level of said Amygdala or an activity level of said at least one related brain region or said at least one biomarker thereof, from said recorded EEG signals, and providing feedback to said population regarding an activity of said Amygdala, or an activity of said at least one related brain region or regarding said at least one biomarker thereof, based on the one or more extracted signals.

Example 19. A method according to any one of examples 15 to 17, wherein said delivered NF treatment is a functional magnetic resonance imaging (fMRI) neurofeedback treatment.

Example 20. A method according to any one of examples 15 to 19, wherein said achieving comprises achieving a reduction of at least 6 points in average in said CAPS-5 scale, up to 3 months from said delivering.

Example 21. A method according to any one of examples 15 to 20, wherein said achieving comprises achieving a reduction of at least 10 points in average in a PTSD Checklist for DSM-5 (PCL-5) scale, after a time period of at least 8 weeks of said delivering, and/or up to 3 months from said delivering.

Example 22. A method according to any one of examples 15 to 21, wherein said selecting comprises selecting a population of female patients diagnosed with chronic PTSD.

Example 23. A method to treat a predetermined patient population for PTSD, the method comprising:
selecting a population of male patients diagnosed with PTSD;
delivering a NF treatment to said population;
achieving a reduction of at least 6 points in average in a CAPS-5 scale in said selected population, after a time period of at least 8 weeks of said delivering.

Example 24. A method according to example 23, wherein said delivering comprises training said selected population using said NF treatment to modulate activation of the Amygdala or activation of at least one related brain region or at least one biomarker thereof.

Example 25. A method according to example 24, wherein said training comprises training said selected population using said NF treatment to modulate said activation, of the Amygdala or activation of said at least one related brain region or said at least one biomarker thereof, when exposed to at least one interface.

Example 26. A method according to example 25, wherein said delivered NF treatment comprises recording EEG signals from said selected population during said exposure to said at least one interface, extracting one or more signals which selectively indicate an activity level of said Amygdala or an activity level of said related brain region or said at least one biomarker thereof, from said recorded EEG signals, and providing feedback to said population regarding an activity of said Amygdala, or regarding an activity of said at least one related brain region or regarding said at least one biomarker thereof, based on the one or more extracted signals.

Example 27. A method according to any one of examples 23 to 25, wherein said delivered NF treatment is a functional magnetic resonance imaging (fMRI) neurofeedback treatment.

Example 28. A method according to any one of examples 23 to 27, wherein said achieving comprises achieving a reduction of at least 6 points in average in said CAPS-5 scale, up to 3 months from said delivering.

Example 29. A method according to any one of examples 23 to 28, wherein said selecting comprises selecting a population of male patients diagnosed with chronic PTSD.

Example 30. A method to treat a predetermined patient population for PTSD, the method comprising:
selecting a population of patients diagnosed with PTSD, wherein said PTSD originates from a non-civilian trauma;
delivering a NF treatment to said selected population;
achieving a reduction of at least 6 points in average in a CAPS-5 scale in said selected population, after a time period of at least 8 weeks of said delivering.

Example 31. A method according to example 30, wherein said delivering comprises training said selected population using said NF treatment to modulate activation of the Amygdala or activation of at least one related brain region or at least one biomarker thereof.

Example 32. A method according to example 31, wherein said training comprises training said selected population using said NF treatment to modulate said activation of the Amygdala or activation of said at least one related brain region or said at least one biomarker thereof, when exposed to at least one interface.

Example 33. A method according to example 32, wherein said delivered NF treatment comprises recording EEG signals from said selected population during said exposure to said at least one interface, extracting one or more signals which selectively indicate an activity level of said Amygdala or an activity level of said at least one related brain region or said at least one biomarker thereof, from said recorded EEG signals, and providing feedback to said population regarding an activity of said Amygdala or an activity of said at least one related brain region or regarding said at least one biomarker thereof, based on the one or more extracted signals.

Example 34. A method according to any one of examples 30 to 32, wherein said delivered NF treatment is a functional magnetic resonance imaging (fMRI) neurofeedback treatment.

Example 35. A method according to any one of examples 30 to 34, wherein said achieving comprises achieving a reduction of at least 6 points in average in said CAPS-5 scale, up to 3 months from said delivering.

Example 36. A method according to any one of examples 30 to 35, wherein said selecting comprises selecting a population of patients diagnosed with chronic PTSD.

Example 37. A method to treat a predetermined patient population for PTSD, the method comprising:
selecting a population of patients diagnosed with PTSD, wherein said PTSD originates from a civilian trauma;
delivering a NF treatment to said selected population;
achieving a reduction of at least 6 points in average in a CAPS-5 scale in said selected population, after a time period of at least 8 weeks of said delivering.

Example 38. A method according to example 37, wherein said delivering comprises training said selected population using said NF treatment to modulate activation of the Amygdala or activation of at least one related brain region or at least one biomarker thereof.

Example 39. A method according to example 38, wherein said training comprises training said selected population using said NF treatment to modulate said activation of the Amygdala or activation of said brain region or said at least one biomarker thereof, when exposed to at least one interface.

Example 40. A method according to example 39, wherein said delivered NF treatment comprises recording EEG signals from said selected population during said exposure to said at least one interface, extracting one or more signals which selectively indicate an activity level of said Amygdala or an activity level of said at least one related brain region or said at least one biomarker thereof, from said recorded EEG signals, and providing feedback to said population regarding an activity of said Amygdala or regarding an activity of said at least one related brain region or regarding said at least one biomarker thereof, based on the one or more extracted signals.

Example 41. A method according to any one of examples 37 to 39, wherein said delivered NF treatment is a functional magnetic resonance imaging (fMRI) neurofeedback treatment.

Example 42. A method according to any one of examples 37 to 41, wherein said achieving comprises achieving a reduction of at least 6 points in average in said CAPS-5 scale, up to 3 months from said delivering.

Example 43. A method according to any one of examples 37 to 42, wherein said achieving comprises achieving a reduction of at least 10 points in average in a PTSD Checklist for DSM-5 (PCL-5) scale, after a time period of at least 8 weeks of said delivering, and/or up to 3 months from said delivering.

Example 44. A method according to any one of examples 37 to 43, wherein said selecting comprises selecting a population of patients diagnosed with chronic PTSD.

Example 45. A method for assessment of a success of a NF treatment in PTSD patients, comprising:
selecting patients diagnosed with PTSD;
delivering a NF treatment to said selected patients;
assessing success of said NF treatment in said patients after a time period between 8 weeks and 12 weeks of said delivering.

Example 46. A method according to example 45, comprising:
prolonging or stopping said delivering based on results of said assessing.

Example 47. A method according to example 45, comprising:
prolonging said delivering and providing an additional treatment to said patient based on results of said assessing.

Example 48. A method according to example 47, wherein said additional treatment comprises at least one of, a pharmaceutical treatment, a psychological treatment, a psychiatric treatment, group therapy and at least one additional NF treatment.

Example 49. A method according to any one of examples 45 to 48, wherein said assessing comprises assessing said success of said NF treatment using at least one of, a CAPS-5 scale, a PTSD Checklist for DSM-5 (PCL-5) scale, a Patient Health Questionnaire (PHQ-9), an Emotion Regulation Questionnaire (ERQ), and/or observation by an expert.

Example 50. A method according to any one of examples 45 to 49, wherein said delivering comprises training said selected patients using said NF treatment to modulate activation of an Amygdala or activation of at least one related brain region or at least one biomarker thereof, when exposed to at least one interface.

Example 51. A method according to example 50, wherein said delivering said NF treatment comprises recording EEG signals from said selected patients during said exposure to said at least one interface, extracting one or more signals which selectively indicate an activity level of said Amygdala or an activity level of said at least one related brain region or said at least one biomarker thereof, from said recorded EEG signals, and providing feedback to said selected patients regarding an activity of said Amygdala or regarding an activity of said at least one related brain region or regarding said at least one biomarker thereof, based on the one or more extracted signals.

Example 52. A method according to example 51, wherein said providing feedback comprises modifying said at least one interface based on the one or more extracted signals indicating said activity level of said Amygdala or said activity level of said at least one related brain region.

Example 53. A method to treat a predetermined patient population for PTSD, the method comprising:
selecting a population of female patients diagnosed with PTSD;
delivering a NF treatment to said population;
wherein said delivering comprises training said selected female population using said NF treatment to modulate activation of the Amygdala or activation of at least one related brain region or at least one biomarker thereof, when exposed to at least one interface.

Example 54. A method according to example 53, wherein said delivering said NF treatment comprises recording EEG signals from said selected population during said exposure to said at least one interface, extracting one or more signals which selectively indicate an activity level of said Amygdala or an activity level of said at least one related brain region or said at least one biomarker thereof, from said recorded EEG signals, and providing feedback to said population regarding an activity of said Amygdala or regarding an activity of said at least one related brain region or regarding said at least one biomarker thereof, based on the one or more extracted signals.

Example 55. A method according to example 54, wherein said providing feedback comprises modifying said at least one interface based on the one or more extracted signals indicating said activity level of said Amygdala or said activity level of said at least one related brain region or said at least one biomarker thereof.

Example 56. A method according to any one of examples 53 to 55, wherein said selecting comprises selecting a population of said female patients diagnosed with chronic PTSD.

Example 57. A method to treat a predetermined patient population for PTSD, the method comprising:
selecting a population of male patients diagnosed with PTSD;
delivering a NF treatment to said population;
wherein said delivering comprises training said selected male population using said NF treatment to modulate activation of the Amygdala or activation of at least one related brain region or at least one biomarker thereof, when exposed to at least one interface.

Example 58. A method according to example 57, wherein said delivering said NF treatment comprises recording EEG signals from said selected population during said exposure to said at least one interface, extracting one or more signals which selectively indicate an activity level of said Amygdala or an activity level of said at least one related brain region or said at least one biomarker thereof, from said recorded EEG signals, and providing feedback to said selected population regarding an activity of said Amygdala or regarding an activity of said at least one related brain region or regarding said at least one biomarker thereof, based on the one or more extracted signals.

Example 59. A method according to example 58, wherein said providing feedback comprises modifying said at least one interface based on the one or more extracted signals indicating said activity level of said Amygdala or said activity level of said at least one related brain region or said at least one biomarker thereof.

Example 60. A method according to any one of examples 57 to 59, wherein said selecting comprises selecting a population of said male patients diagnosed with chronic PTSD.

Example 61. A method to treat a predetermined patient population for PTSD, the method comprising:
selecting a population of patients diagnosed with PTSD originating from non-civilian trauma;
delivering a NF treatment to said selected population;
wherein said delivering comprises training said selected population using said NF treatment to modulate activation of the Amygdala or activation of at least one related brain region or at least one biomarker thereof, when exposed to at least one interface.

Example 62. A method according to example 61, wherein said delivering said NF treatment comprises recording EEG signals from said selected population during said exposure to said at least one interface, extracting one or more signals which selectively indicate an activity level of said Amygdala or an activity level of said at least one related brain region or said at least one biomarker thereof, from said recorded EEG signals, and providing feedback to said selected population regarding an activity of said Amygdala or regarding an activity of said at least one related brain region or regarding said at least one biomarker thereof, based on the one or more extracted signals.

Example 63. A method according to example 62, wherein said providing feedback comprises modifying said at least one interface based on the one or more extracted signals indicating said activity level of said Amygdala or said activity of said at least one related brain region or said at least one biomarker thereof.

Example 64. A method according to any one of examples 61 to 63, wherein said selecting comprises selecting a population of said patients diagnosed with chronic PTSD.

Example 65. A method to treat a predetermined patient population for PTSD, the method comprising:
selecting a population of patients diagnosed with PTSD originating from civilian trauma;
delivering a NF treatment to said selected population;
wherein said delivering comprises training said selected population using said NF treatment to modulate activation of the Amygdala or activation of at least one related brain region or at least one biomarker thereof, when exposed to at least one interface.

Example 66. A method according to example 65, wherein said delivering said NF treatment comprises recording EEG signals from said selected population during said exposure to said at least one interface, extracting one or more signals which selectively indicate an activity level of said Amygdala or an activity level of said at least one related brain region or said at least one biomarker thereof, from said recorded EEG signals, and providing feedback to said selected population regarding an activity of said Amygdala or regarding an activity of said at least one related brain region or regarding said at least one biomarker thereof, based on the one or more extracted signals.

Example 67. A method according to example 66, wherein said providing feedback comprises modifying said at least one interface based on the one or more extracted signals indicating said activity level of said Amygdala or an activity level of said at least one related brain region or said at least one biomarker thereof.

Example 68. A method according to any one of examples 65 to 67, wherein said selecting comprises selecting a population of said patients diagnosed with chronic PTSD.

Example 69. A system for delivering of a neurofeedback treatment for treating PTSD, comprising:
at least one user interface, configured to deliver at least one indication;
a control unit, comprising:
a memory, wherein said memory stores at least one protocol of a neurofeedback (NF) treatment or indications thereof;
and a control circuitry, wherein said control circuitry is configured to deliver said NF treatment to a PTSD patient based on said at least one protocol or indications thereof stored in said memory, and to signal said at least user interface to generate an indication with information to perform an assessment of results of said NF treatment within a time period between 8 weeks and 12 weeks from initiating the delivery of said NF treatment.

Example 70. A system according to example 69, wherein said control circuitry is configured to receive an input signal with information regarding results of said assessment and to signal said at least one user interface to generate an indication with information which includes a suggestion to modify said at least one NF treatment protocol or to stop the delivery of said NF treatment according to the results of said assessment.

Example 71. A system according to example 69, wherein said memory stores a plurality of protocols of said NF treatment, and wherein said control circuitry is configured to receive an input signal with information regarding results of said assessment and to signal said at least one user interface to generate an indication with information which includes a suggestion to replace said at least one protocol of said NF treatment with a different protocol of said plurality of protocols, according to the results of said assessment.

Example 72. A system according to example 69, wherein said system comprises at least one remote device storing a plurality of protocols of said NF treatment, and wherein said control unit comprises a communication circuitry configured to communicate with said at least one remote device, and wherein said control circuitry is configured to receive an input signal with information regarding results of said assessment and to signal said at least one user interface to generate an indication with information which includes a suggestion to replace said at least one protocol of said NF treatment with a different protocol of said plurality of protocols stored in said at least one remote device, according to the results of said assessment.

Example 73. A system according to example 72, wherein said at least one remote device comprises at least one of, a remote computer, a server, a cloud storage device, and a cloud storage and processing device.

Example 74. A system according to any one of examples 69 to 73, wherein said control unit comprises an EEG recording unit configured to record electrical signals from one or more electrodes positioned on a head of a subject, and a patient interface;
wherein said control circuitry is configured to:
signal said patient interface to present an interface stored in said memory to said PTSD patient, wherein said interface is selected to increase activity of the Amygdala or activity of at least one related brain region or at least one biomarker thereof, in said subject;
measure EEG signals according to said electrical signals received by said EEG recording unit;
detect a change in activity of said Amygdala or in activity of said at least one related brain region or in said at least one biomarker thereof using said measured EEG signals and at least one algorithm and/or an electrical fingerprint (EFP) of said Amygdala or of said at least one related brain region or said at least one biomarker thereof, stored in said memory; and
modify said presentation of said interface to said PTSD patient according to said detected change in activity.

Example 75. A method for assessing success of a NF treatment in a PTSD patient, comprising:
receiving results of at least one assessment of a PTSD patient performed following initiation of a NF treatment in said patient;
determining a relation between said assessment results and expected assessment results;
generating an indication based on said determine relation.

Example 76. A method according to example 75, wherein said determining comprises determining that the assessment results are lower than said expected results, and wherein said generating comprises generating said indication with instructions to modify at least one parameter of said NF treatment and to deliver at least one additional NF treatment session of said NF treatment using said modified at least one NF treatment parameter.

Example 77. A method according to example 76, wherein said at least one NF treatment parameter comprises, duration of a NF treatment session, number of additional NF treatment sessions, type and/or content of a visual interface presented to the patient during a NF treatment session, analysis performed to EEG signals recorded from the subject brain during a NF treatment session, said analysis is performed in order to detect modulation of activity of at least one of, limbic network, at least one limbic brain region and an amygdala brain region based on EEG signals alone.

Example 78. A method according to any one of examples 76 or 77, wherein said generated indication comprise instructions to combine said NF treatment with said modified parameter with at least one different treatment.

Example 79. A method according to example 75, wherein said determining comprises determining that the assessment results are lower than said expected results, and wherein said generating comprises generating said indication with instructions to replace said NF treatment with at least one different treatment, or to continue said NF treatment in combination with said at least one different treatment.

Example 80. A method according to any one of examples 78 or 79, wherein said at least one different treatment comprises at least one of, a cognitive behavioral treatment (CBT), a psychological treatment, a psychiatric treatment, and/or a drug treatment.

Example 81. A method according to example 75, wherein said determining comprises determining that the assessment results are lower than said expected results, and wherein said generating comprises generating said indication with instructions to deliver one or more additional NF treatment sessions, or to stop the delivery of said NF treatment.

Example 82. A method according to any one of examples 75 to 80, wherein said at least one assessment comprises a Clinician-Administered PTSD Scale for DSM-5 (CAPS-5) scale, and wherein said expected assessment results comprise a reduction of at least 6 points in said CAPS-5 scale in said patient after at least 8 weeks of NF treatment.

Example 83. A method according to any one of examples 75 to 82, wherein said at least one assessment comprises a PTSD Checklist for DSM-5 (PCL-5) scale, and wherein said expected results comprise a reduction of at least 9 points in said PCL-5 scale after at least 8 weeks of NF treatment.

Example 84. A method according to any one of examples 75 to 83, comprises delivering said generated indication to a supervisor of said NF treatment in said patient.

Example 85. A system for assessing success of a NF treatment comprising:
a memory, wherein said memory stores results of at least one assessment of a PTSD patient performed following initiation of said NF treatment in said PTSD patient, and expected results of said assessment;
a control circuitry, wherein said control circuitry is configured to determine a relation between said stored results of said at least one assessment and said stored expected results, and to generate an indication according to said determined relation.

Example 86. A system according to example 85, wherein said control circuitry is configured to determine that the assessment results are lower than said expected results, and to generate said indication with instructions to modify at least one parameter of said NF treatment, and to deliver to said patient at least one additional NF treatment session of said NF treatment using said modified at least one NF treatment parameter.

Example 87. A system according to example 86, wherein said indication generated by said control circuitry includes instructions to modify said at least one NF treatment parameter which comprises at least one of, duration of a NF treatment session, number of additional NF treatment sessions, type and/or content of a visual interface presented to the patient during a NF treatment session, analysis performed to EEG signals recorded from the subject brain during a NF treatment session, said analysis is performed in order to detect modulation of activity of at least one of, limbic network, at least one limbic brain region and an amygdala brain region based on EEG signals alone.

Example 88. A system according to any one of examples 86 or 87, wherein said generate indication comprises instructions to combine said NF treatment including said modified parameter, with at least one additional treatment.

Example 89. A system according to example 85, wherein said control circuitry is configured to determine that the assessment results are lower than said expected results, and generate said indication with instructions to replace said NF treatment with at least one additional treatment, or to continue said NF treatment in combination with said at least one additional treatment.

Example 90. A system according to any one of examples 88 or 89, wherein said at least one additional treatment comprises at least one of, a cognitive behavioral treatment (CBT), a psychological treatment, a psychiatric treatment, and/or a drug treatment.

Example 91. A system according to example 85, wherein said control circuitry is configured to determine that the assessment results are lower than said expected results, and to generate said indication with instructions to deliver one or more additional NF treatment sessions, or to stop the delivery of said NF treatment.

Example 92. A system according to any one of examples 85 to 91, wherein said assessment results stored in said memory comprise results of a Clinician-Administered PTSD Scale for DSM-5 (CAPS-5) scale, wherein said expected assessment results stored in said memory comprise a reduction of at least one 6 points in average in said CAPS-5 scale in said patients after at least 8 weeks of said NF treatment.

Example 93. A system according to any one of examples 85 to 92, wherein said assessment results stored in said memory comprise results of a PTSD Checklist for DSM-5 (PCL-5) scale, and wherein said expected assessment results stored in said memory comprise a reduction of at least one 9 points in average in said PCL-5 scale in said patients after at least 8 weeks of said NF treatment.

Example 94. A system according to any one of examples 85 to 93, wherein said system comprises a communication circuitry, and wherein said system receives said results of said at least one assessment using said communication circuitry, and wherein said control circuitry signals said communication circuitry to deliver said generated indication to a remote device.

Example 95. A method for assessing success of a NF treatment in a group of PTSD patients, comprising:
receiving results of at least one assessment performed in a group of PTSD patients following initiation of said NF treatment is said patients;
determining a relation between said assessment results and expected assessment results;
generating an indication based on said determined relation.

Example 96. A method according to example 95, comprising processing said received results to generate separate assessment results for at least one subclass of said group of PTSD patients, and wherein said determining comprises determining a relation between said separate assessment results of said at least one subclass of PTSD patients and said expected assessment results.

Example 97. A method according to example 96, wherein said at least one subclass of said PTSD patients comprises at least one of, female PTSD patients, male PTSD patients, PTSD patients in which said PTSD originates from a civilian trauma and PTSD patients in which said PTSD originates from a non-civilian trauma.

Example 98. A method according to any one of examples 96 or 97, wherein said determining comprises determining that said assessment results of said at least one subclass is lower than said expected results, and wherein said generating comprises generating an indication with instructions comprising at least one of, instructions to modify at least one parameter of said NF treatment, instructions to replace an existing protocol of said NF treatment with a different protocol, instructions to stop said NF treatment, and instructions to combine the NF treatment with at least one additional treatment.

Example 99. A method according to any one of examples 95 to 98, wherein said at least one assessment comprises at least one of, a PTSD Checklist for DSM-5 (PCL-5) scale, and a Clinician-Administered PTSD Scale for DSM-5 (CAPS-5) scale.

Example 100. A system for assessing success of a NF treatment comprising:
a memory, wherein said memory stores results of at least one assessment performed in a group of PTSD patients following initiation of said NF treatment in said PTSD patients, and expected results of said assessment;
a control circuitry, wherein said control circuitry is configured to determine a relation between said stored results of said at least one assessment and said stored expected results, and to generate an indication according to said determined relation.

Example 101. A system according to example 100, wherein said control circuitry is configured to process said received results to generate separate assessment results for at least one subclass of said group of said PTSD patients and to determine a relation between said separate assessment results of said at least one subclass of PTSD patients and said expected assessment results.

Example 102. A system according to example 101, wherein said at least one subclass of said PTSD patients comprises at least one of, female PTSD patients, male PTSD patients, PTSD patients in which said PTSD originates from a civilian trauma and PTSD patients in which said PTSD originates from a non-civilian trauma.

Example 103. A system according to any one of examples 101 or 102, wherein said determining comprises determining that said assessment results of said at least one subclass is lower than said expected results, and to generate an indication with instructions comprising at least one of, instructions to modify at least one parameter of said NF treatment, instructions to replace an existing protocol of said NF treatment with a different protocol, instructions to stop said NF treatment, and instructions to combine the NF treatment with at least one additional treatment.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as assessing biomarker values in real time and controlling the behavior of a digital or other interactive interface in real time, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a general flow chart of a PTSD neurofeedback (NF) treatment process, according to some exemplary embodiments of the invention;

FIG. 2 is a flow chart of a NF treatment session, according to some exemplary embodiments of the invention;

FIG. 3 is a block diagram of a system for delivery of PTSD NF treatment, according to some exemplary embodiments of the invention;

FIG. 4A is a flow chart of a PTSD NF treatment process which includes assessment of a state of the patient between 8 weeks to 12 weeks from initiating the delivery of NF treatment sessions, according to some exemplary embodiments of the invention;

FIG. 4B is a flow chart of a process for selecting a sub-population of female subjects for a NF treatment, according to some exemplary embodiments of the invention;

FIG. 4C is a flow chart of a process for selecting for the NF treatment a sub-population of patients diagnosed with PTSD originating from civilian trauma, according to some exemplary embodiments of the invention;

FIG. 5A is a flow chart of a PTSD NF treatment process performed in a clinical study and according to some exemplary embodiments of the invention;

FIG. 5B is a schematic illustration showing the use of an electrical fingerprint (EFP) model in determining an activity level of the Amygdala, during the PTSD NF treatment, as performed in the clinical study and according to some exemplary embodiments of the invention;

FIG. 5C is a schematic block diagram of a device for assessment of an effect of a NF treatment on a state of a patient receiving the treatment, according to some exemplary embodiments of the device;

FIG. 5D is a flow chart of a general assessment process, according to some embodiments of the invention.

FIG. 5E is a flow chart of a detailed assessment process, according to some exemplary embodiments of the invention;

FIG. 5F is a flow chart of a process for assessment of an effect of a NF treatment on a specific population of patients, according to some exemplary embodiments of the invention;

FIG. 6A is a graph showing an average change in a score of a Clinician-Administered PTSD Scale for DSM-5 (CAPS-5) scale during the clinical study;

FIG. 6B is a graph showing an average change in a score of Criterion B of a CAPS-5 scale during the clinical study;

FIG. 6C is a graph showing an average change in a score of Criterion C of a CAPS-5 scale during the clinical study;

FIG. 6D is a graph showing an average change in a score of Criterion D of a CAPS-5 scale during the clinical study;

FIG. 6E is a graph showing an average change in a score of Criterion E of a CAPS-5 scale during the clinical study;

FIG. 6F is a graph showing an average change in a score of Criterion G of a CAPS-5 scale during the clinical study;

FIG. 7A is a graph showing an average change in a CAPS-5 scale score during the clinical study, in patients with PTSD that originates from civilian trauma;

FIG. 7B is a graph showing an average change in a score of Criterion B of a CAPS-5 scale during the clinical study, in patients with PTSD that originates from civilian trauma;

FIG. 7C is a graph showing an average change in a score of Criterion C of a CAPS-5 scale during the clinical study, in patients with PTSD that originates from civilian trauma;

FIG. 7D is a graph showing an average change in a score of Criterion D of a CAPS-5 scale during the clinical study, in patients with PTSD that originates from civilian trauma;

FIG. 7E is a graph showing an average change in a score of Criterion E of a CAPS-5 scale during the clinical study, in patients with PTSD that originates from civilian trauma;

FIG. 7F is a graph showing an average change in a score of Criterion G of a CAPS-5 scale during the clinical study, in patients with PTSD that originates from civilian trauma;

FIG. 8A is a graph showing an average change in a PCL-5 scale score during the clinical study;

FIG. 8B is a graph showing an average change in a PCL-5 scale score during the clinical study, in patients with PTSD that originates from civilian trauma;

FIG. 9A is a graph showing an average change in a score of a PHQ-9 scale during the clinical study; and

FIG. 9B is a graph showing an average change in a score of a PHQ-9 scale during the clinical study, in patients with PTSD that originates from civilian trauma.

### DESCRIPTION OF SPECIFIC EMBODIMENTS OF THE INVENTION

The present invention, in some embodiments thereof, relates to a stress disorder treatment and, more particularly, but not exclusively, to a post-traumatic stress disorder (PTSD) treatment.

An aspect of some embodiments relates to achieving a reduction of at least 6 points in a Clinician-Administered PTSD Scale for DSM-5 (CAPS-5), following a neurofeedback (NF) treatment delivered to human subjects during a time-period in a range between 8 consecutive weeks and 12 consecutive weeks. In some embodiments, the reduction of the at least 6 points is achieved in human subjects diagnosed with PTSD treated with the NF treatment. In some embodiments, the reduction in the at least 6 points of the CAPS-5 scale is achieved following a NF treatment, also termed herein as a NF training, that lasted between 8 weeks and 12 weeks, for example between 8 consecutive weeks and 12 consecutive weeks. In some embodiments, the reduction in CAPS-5 of at least 6 points, comprises a reduction of at least 7 points, a reduction of at least 10 points, a reduction of at least 15 points, a reduction of at least 20 points, or any intermediate, smaller or larger value.

According to some exemplary embodiments, In some embodiments, the human subjects are diagnosed with chronic PTSD, for example human subjects having PTSD symptoms for at 1 months for example for at least 6 months, for at least 10 months, for at least 12 months, for at least 14 months, for at least 16 months, for at least 24 months, or any intermediate, smaller or larger time period. Alternatively or additionally, the human subjects are diagnosed with chronic PTSD according to the Diagnostic and Statistical Manual of Mental Disorders, Fifth Edition (DSM-V) or any earlier or later editions.

According to some embodiments, the human subjects are female subjects. Alternatively or additionally, the human subjects are diagnosed with PTSD originating from a civilian trauma.

According to some exemplary embodiments, the NF treatment comprises monitoring the activity or at least one biomarker associated with the activity, a of at least one limbic brain region, for example the Amygdala and delivery of a signal to the subject based on the monitored activity. In some embodiments, the activity of the at least one limbic brain region is monitored using at least one EEG signal recorded from electrodes positioned on a head of the subject, for example as described in international patent application publication no. WO2012104853A2, filed on 2 Feb. 2012, and as described in international patent application publication no. WO2020121299A1, filed on 9 Dec. 2019, the contents of which is incorporated as a reference in their entirety.

According to some exemplary embodiments, the NF treatment is performed in a clinic or an office outside the subject home, and/or at the subject home. In some embodiments, the NF treatment comprises at least two treatment sessions per week.

An aspect of some embodiments relates to treating human subjects diagnosed with PTSD with a NF treatment during a time period between 8 weeks and 12 weeks, for example during a time period between 8 weeks and 10 weeks, during a time period between 8 weeks and 11 weeks, during a time period between 8 weeks and 9 weeks, during a time period between 10 weeks and 12 weeks, or any intermediate, shorter or longer time period. In some embodiments, a reduction of at least 6 points in a Clinician-Administered PTSD Scale for DSM-5 (CAPS-5), is achieved up to 3 months from the end of the NF treatment, for example from the last treatment session of the NF treatment. In some embodiments, the reduction in CAPS-5 is achieved up to 2 months, up to 1 months, between 1 month and 3 months, or any intermediate, shorter or longer time period from the end of the NF treatment.

According to some exemplary embodiments, a reduction of at least 6 points of the CAPS-5 scale is achieved at the end of the NF treatment, for example up to 2 months from the last treatment session of the NF treatment. In some embodiments, the reduction of the at least 6 points of the CAPS-5 scale is achieved when finishing the delivery of the NF treatment, for example after the time period of 8-12 weeks of the delivery of the NF treatment. 5

According to some exemplary embodiments, the human subjects are diagnosed with chronic PTSD, for example human subjects having PTSD symptoms for at 1 months for example for at least 6 months, for at least 10 months, for at least 12 months, for at least 14 months, for at least 16 months, for at least 24 months, or any intermediate, smaller or larger time period. Alternatively or additionally, the human subjects are diagnosed with chronic PTSD according to 10 the Diagnostic and Statistical Manual of Mental Disorders, Fifth Edition (DSM-V) or any earlier or later editions.

An aspect of some embodiments relates to achieving a reduction of at least 6 points in a Clinician-Administered PTSD Scale for DSM-5 (CAPS-5), following a neurofeedback (NF) treatment delivered to human subjects during a time-period of at least 8 weeks. In some embodiments, the reduction of the at least 6 points is achieved in a population of female human subjects diagnosed with PTSD treated with the NF treatment. In some embodiments, the population of female human subjects are selected for the NF treatment, for example based on a predicted success in reducing CAPS-5 in at least 6 points after 8 weeks of NF treatment in the selected population. In some embodiments, the female human subjects are diagnosed with chronic PTSD, for example human subjects having PTSD symptoms for at least 1 months, for example for at least 14 months, for at least 16 months, for at least 24 months, or any intermediate, smaller or larger time period. Alternatively or additionally, the female human subjects are diagnosed with chronic PTSD according to the Diagnostic and Statistical Manual of Mental Disorders, Fifth Edition (DSM-V) or any earlier or later edition. Optionally, the female human subjects are diagnosed with PTSD originating from at least one civilian trauma event, for example a sexual trauma, an accident and violence, or with PTSD originating from a non-civilian trauma.

An aspect of some embodiments relates to achieving a reduction of at least 6 points in a Clinician-Administered PTSD Scale for DSM-5 (CAPS-5), following a neurofeedback (NF) treatment delivered to human subjects during a time-period of at least 8 weeks. In some embodiments, the reduction of the at least 6 points is achieved in a population of male human subjects diagnosed with PTSD treated with the NF treatment. In some embodiments, the male human subjects population is selected for the NF treatment, for example based on a predicted success in reducing CAPS-5 in at least 6 points after 8 weeks of NF treatment in the selected population. In some embodiments, the male human subjects are diagnosed with chronic PTSD, for example human subjects having PTSD symptoms for at least 1 months, for example for at least 14 months, for at least 16 months, for at least 24 months, or any intermediate, smaller or larger time period. Alternatively or additionally, the female human subjects are diagnosed with chronic PTSD according to the Diagnostic and Statistical Manual of Mental Disorders, Fifth Edition (DSM-V) or any earlier or later edition. Optionally, the male human subjects are diagnosed with PTSD originating from at least one civilian trauma event, for example a sexual trauma, an accident and violence, or with PTSD originating from non-civilian trauma.

An aspect of some embodiments relates to achieving a reduction of at least 6 points in a Clinician-Administered PTSD Scale for DSM-5 (CAPS-5), following a neurofeedback (NF) treatment delivered to human subjects during a time-period of at least 8 weeks. In some embodiments, the reduction of the at least 6 points is achieved in a population of human subjects diagnosed with PTSD originating from civilian trauma, treated by the NF treatment. In some embodiments, the population of human subject is selected for the NF treatment, for example based on a predicted success in reducing CAPS-5 in at least 6 points after 8 weeks of NF treatment in the selected population. In some embodiments, the human subjects are diagnosed with chronic PTSD, for example human subjects having PTSD for at least 1 month, for example for at least 14 months, for at least 16 months, for at least 24 months, or any intermediate, smaller or larger time period. Alternatively or additionally, the human subjects are diagnosed with chronic PTSD according to the Diagnostic and Statistical Manual of Mental Disorders, Fifth Edition (DSM-V).

An aspect of some embodiments relates to achieving a reduction of at least 6 points in a Clinician-Administered PTSD Scale for DSM-5 (CAPS-5), following a neurofeedback (NF) treatment delivered to human subjects during a time-period of at least 8 weeks. In some embodiments, the reduction of the at least 6 points is achieved in a population of human subjects diagnosed with PTSD originating from non-civilian trauma, treated by the NF treatment. In some embodiments, the population of human subjects is selected for the NF treatment, for example based on a predicted success in reducing CAPS-5 in at least 6 points after 8 weeks of NF treatment in the selected population. In some embodiments, the human subjects are diagnosed with chronic PTSD, for example human subjects having PTSD for at least 1 month, for example for at least 14 months, for at least 16 months, for at least 24 months, or any intermediate, smaller or larger time period. Alternatively or additionally, the human subjects are diagnosed with chronic PTSD according to the Diagnostic and Statistical Manual of Mental Disorders, Fifth Edition (DSM-V).

An aspect of some embodiments relates to delivering a NF treatment to a selected population of female patients diagnosed with PTSD. In some embodiments, the delivering of NF treatment comprises training the selected female population using the NF treatment to modulate activation of the Amygdala when exposed to at least one interface, for example a stressor. In some embodiments, the population of patients is selected for the NF treatment, for example based on a predicted success in reducing CAPS-5 in at least 6 points after at least 8 weeks of NF treatment, and up to 3 months following the completion of the NF treatment, in the selected population, as demonstrated for example in Tables A and B, and in the results of the clinical study. In some embodiments, the at least one interface comprises a visual and/or an audio interface. Optionally, the at least one interface is selected to increase a stress feeling in a subject when presented to the subject.

An aspect of some embodiments relates to delivering a NF treatment to a selected population of male patients diagnosed with PTSD. In some embodiments, the delivering of NF treatment comprises training the selected male population using the NF treatment to modulate activation of the Amygdala when exposed to at least one interface. In some embodiments, the population of patients is selected for the NF treatment, for example based on a predicted success in reducing CAPS-5 in at least 6 points after 8 weeks of NF treatment in the selected population.

An aspect of some embodiments relates to delivering a NF treatment to a selected population of patients diagnosed with PTSD which originates from non-civilian trauma. In some embodiments, the delivering of NF treatment comprises training the selected population using the NF treatment to modulate activation of the Amygdala when exposed to at least one interface. In some embodiments, the population of patients is selected for the NF treatment, for example based on a predicted success in reducing CAPS-5 in at least 6 points after 8 weeks of NF treatment in the selected population.

An aspect of some embodiments relates to delivering a NF treatment to a selected population of patients diagnosed with PTSD which originates from a civilian trauma. In some embodiments, the delivering of NF treatment comprises training the selected population using the NF treatment to modulate activation of the Amygdala when exposed to at least one interface. In some embodiments, the population of patients is selected for the NF treatment, for example based on a predicted success in reducing CAPS-5 in at least 6 points after 8 weeks of NF treatment in the selected population.

An aspect of some embodiments relates to evaluating, for example assessing, results of a NF treatment used for treating human subjects diagnosed with PTSD, after a time period between 8 consecutive weeks to 12 consecutive weeks from the initiation of the NF treatment. In some embodiments, results of the NF treatment are evaluated using CAPS-5. Alternatively or additionally, the results of the NF treatment are evaluated using one or more CAPS-5 criteria. Alternatively or additionally, the results of the NF treatment are evaluated using a PTSD Checklist for DSM-5 (PCL-5). Alternatively or additionally, the results of the NF treatment are evaluated using a Patient Health Questionnaire-9 (PHQ-9).

According to some embodiments, a treatment regime provided to the PTSD subject, which includes the NF treatment, is modified based on the evaluation results. In some embodiments, modifying the treatment regime comprises continuing the NF treatment for one or more additional NF treatment sessions, or one or more additional weeks. Alternatively, modifying the treatment regime comprises stopping the NF treatment. Alternatively, modifying the treatment regime comprises replacing or combining the NF treatment with at least one different treatment, for example a pharmaceutical treatment, a psychological treatment, and a group therapy. Alternatively, modifying treatment regime comprises adding future NF treatment sessions.

An aspect of some embodiments relates to a system for delivery of a NF treatment that is configured to deliver an indication with information to perform an assessment of a success of the NF treatment at least 8 weeks from initiating the NF treatment. In some embodiments, the system is configured to deliver an indication with information to perform the assessment within a time period between 8 weeks and 12 weeks from initiating the NF treatment.

According to some embodiments, the system, for example a control circuitry of the system, is configured to receive input with results of the assessment and to provide an indication with information how to modify an existing NF treatment protocol according to the assessment results. Alternatively, the system is configured to provide an indication with a suggestion to replace the existing NF treatment protocol with a different NF treatment protocol, according to the assessment results. Alternatively, the system is configured to provide an indication with a suggestion to stop or prolong the delivery of the existing NF treatment protocol, according to the assessment results.

Alternatively, the system is configured to provide an indication with a suggestion to combine the delivery of the existing NF treatment protocol with an additional therapy, according to the assessment results. In some embodiments, the additional therapy comprises at least one of, pharmaceutical therapy, psychological therapy, psychiatric therapy, and group therapy.

According to some embodiments, a NF treatment delivered to PTSD patients comprises, presenting at least one interface to a patient. In some embodiments, the at least one interface is selected to increase an activity of the Amygdala or at least one related brain region, for example at least one brain region of the limbic system, or at least one biomarker thereof, for example at least one biomarker of the amygdala activity or of the activity of the at least one related brain region. In some embodiments, the at least one biomarker comprises a physiological marker, for example heart rate, blood pressure, skin conductance, and sweat secretion. In some embodiments, the at least one biomarker comprises the at least one electrical fingerprint (EFP), for example an EFP model of at least one limbic brain region, for example the EFP model described in for example as described in international patent application publication no. WO2012104853A2, filed on 2 Feb. 2012, and as described in international patent application publication no. WO2020121299A1, filed on 9 Dec. 2019, the contents of which is incorporated as a reference in their entirety. In some embodiments, the at least one interface is visually presented to the patient, optionally in combination with an audio signal. In some embodiments, EEG signals are recorded from the brain of the subject using one or more electrodes located on the head of the subject. In some embodiments, the recorded EEG signals are then processed using at least one algorithm and/or at least one EFP to extract signals or indications thereof, indicating an activity of the Amygdala or activity of at least one related brain region, or a biomarker thereof. In some embodiments, the EEG signals are recorded and processed while the patient is exposed to the at least one interface.

According to some embodiments, the system provides a feedback to the patient indicating an activity level of the Amygdala or an activity level of at least one related brain region, based on the extracted signals. Optionally, the feedback is provided by modifying, for example online, the at least one interface presented to the patient while the patient is exposed to the at least one interface. As used herein, the term “online” means in a delay of up to 20 seconds, in a delay of up to 15 seconds, in a delay of up to 10 seconds, in a delay of up to 5 seconds, in a delay of up to 2 seconds, or any intermediate, shorter or longer delay from recording the EEG signals.

According to some embodiments, the at least one interface comprises a visual and/or an audio interface presented to the patient using at least one of a display, a virtual reality device, an augmented reality device, or any other visualization device.

According to some exemplary embodiments, the NF treatment, for example the NF training, is delivered to a patient without a need to use a fMRI device.

An aspect of some embodiments relates to assessing success of a NF treatment on a subject after initiating the NF treatment, for example after at least one session of the NF treatment or after at least 1 week of the NF treatment. In some embodiments, the assessment is based on results of at least one assessment scale measured after the at least one session or after at least 1 week from initiating the NF treatment. In some embodiments, the treatment success assessment is based on a relation between the measured results of the assessment scale and stored results. In some embodiments, the stored results comprise previous results of the assessment scale previously measured in the same subject. Alternatively or additionally, the stored results comprise target results indicating a target success level of the NF treatment.

According to some embodiments, the NF treatment or overall treatment in the specific subject is modified based on the determined ratio between the measured results of the assessment scale and stored results or indications thereof. In some embodiments, modifying the NF treatment comprises modifying at least one parameter of the NF treatment, and optionally delivering at least one additional NF treatment session to the subject or to a different subject with the modified parameter. Alternatively, modifying the NF treatment comprises stopping the NF treatment or combining the NF treatment with at least one additional treatment.

According to some embodiments, the success assessment is performed by a device, optionally communicating with a different device which delivers the NF treatment. Alternatively, the assessment is performed by the same device or system delivering the NF treatment. In some embodiments, the device automatically modifies the treatment delivered to the subject or to a different subject. Alternatively, the device generates at least one indication with suggestions, for example recommendations, how to modify the treatment. In some embodiments, the assessment scale comprises at least one of, CAPS-5, PCL-5, and PHQ-9, and the measured results include scores of the assessment scale.

An aspect of some embodiments relates to assessing success of a NF treatment in a group of subjects after initating a NF treatment, for example at least one session of the NF treatment or after at least 1 week of the NF treatment. In some embodiments, the assessment is based on results of at least one assessment scale conducted by the group of subjects after initiating the NF treatment. In some embodiments, the treatment success assessment is based on a determined relation between the results of the assessment scale and stored results. In some embodiments, the stored results comprise previous results of the assessment scale previously measured in the same and/or subjects. Alternatively or additionally, the stored results comprise target results indicating a target success level of the NF treatment.

According to some embodiments, based on the determined relation, a NF treatment for one or more subjects belonging to the group of subjects is modified. Alternatively or additionally, based on the determined relation, the results of the assessment scale of the group of subjects are further processed, for example classified, to isolate results of one or more sub-population of subjects includes in the group of subjects. In some embodiments, a relation between the results of the sub-population of subjects and stored results is determined, for example to identify if the specific sub-population performed better in the assessment scale relative to the overall larger group of subjects. In some embodiments, a NF treatment for one or more subjects belonging to the specific sub-population is modified, for example to increase the effect of the NF treatment in subjects of the specific sub-population.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary General Process for PTSD Treatment

A subject diagnosed with PTSD is treated using NF treatment for several weeks, in order to achieve reduction in a score of a CAPS-5 scale following the treatment. In some embodiments, the achieved reduction indicates a significant clinical improvement of a PTSD state.

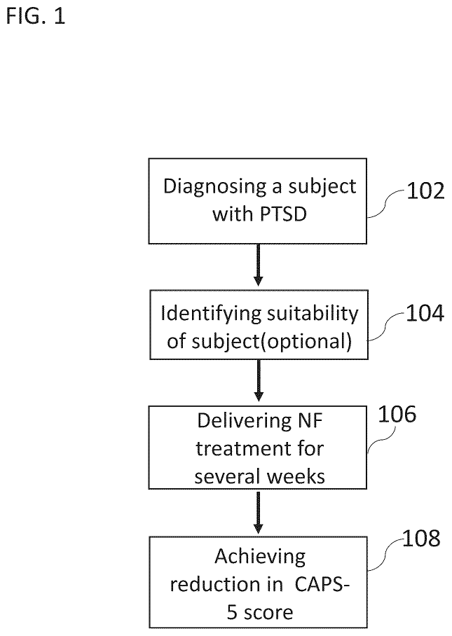

Reference is now made to FIG. 1, depicting a process for treating a subject diagnosed with PTSD using NF, according to some exemplary embodiments of the invention.

According to some exemplary embodiments, a subject is diagnosed with a stress disorder, for example PTSD, at block 102. In some embodiments, the subject is diagnosed with PTSD according to at least one criteria of the DSM-V. Optionally, the subject is diagnosed with chronic PTSD, for example, the subject experiences one or more PTSD symptom for a period of at least 1 month for example at least 14 months ago, at least 18 months ago, at least 24 months ago or any intermediate, shorter or longer time period from initially experiencing PTSD symptoms. In some embodiments, the diagnosis is performed using one or more diagnostic tools, for example questionnaires. Alternatively or additionally, the diagnosis is performed based on observation and/or an expert experience.

According to some exemplary embodiments, the trauma event is a non-civilian trauma event, for example a trauma event occurred during combat activity, or as a response to combat activity.

According to some exemplary embodiments, suitability of a subject for a NF treatment is optionally identified at block 104. In some embodiments, identifying subject suitability comprises identifying a potential of the subject to achieve reduction in a score of a CAPS-5 scale following the NF treatment. In some embodiments, identifying subject suitability comprises identifying a potential of a subject to achieve reduction of at least 6 points for example at least 6 points, at least 10 points or any intermediate, smaller or larger value, in a CAPS-5 scale following a NF treatment, of at least 8 weeks, for example at least 8 consecutive weeks. Alternatively or additionally, identifying subject suitability comprises identifying a potential of a subject to achieve reduction of at least 6 points in a CAPS-5 scale following a NF treatment that lasts between 8 consecutive weeks and 12 consecutive weeks.

According to some exemplary embodiments, identifying subject suitability for the NF treatment at block 104 comprises, identifying that the subject is a female subject. Alternatively or additionally, identifying subject suitability for the NF treatment comprises identifying that a PTSD state of the subject originates from a non-non-civilian trauma event.

According to some exemplary embodiments, a NF treatment is delivered to the subject, at block 106. In some embodiments, the NF treatment is delivered during several consecutive weeks. In some embodiments, the NF treatment is delivered during a time period of at least 8 consecutive weeks, for example at least 10 consecutive weeks, at least 12 consecutive weeks, at least 15 consecutive weeks, or any intermediate, smaller or larger time period. In some embodiments, the NF treatment is delivered during a time period between 8 consecutive weeks and 12 consecutive weeks, for example during a time period between 8 consecutive weeks and 9 consecutive weeks, during a time period between 8 consecutive weeks and 10 consecutive weeks, during a time period between 9 consecutive weeks and 11 consecutive weeks, or any intermediate, smaller or larger time period.

According to some exemplary embodiments, the subject achieves a reduction in CAPS-5 score following the NF treatment, at block 108. In some embodiments, the subject achieves a reduction of at least 6 points, for example at least 6 points, at least 10 points, at least 15 points or any intermediate, smaller or larger reduction compared to a CAPS-5 score measured prior to the NF treatment. In some embodiments, the subject achieves a reduction of at least 6 points points in the CAPS-5 scale after a NF treatment that lasts between 8 weeks and 12 weeks, where optionally the final treatment session is at the last week.

### Exemplary Treatment Session

According to some exemplary embodiments, a NF treatment for treating PTSD comprises a plurality of treatment sessions. In some embodiments, the treatment sessions are performed during at least 8 consecutive weeks. In some embodiments, the treatment sessions are performed during a total time period of 8 to 12 weeks, for example 8 to 12 consecutive weeks. In some embodiments, the NF treatment comprises at least 15 treatment sessions performed during a time period of 8 to 12 weeks. In some embodiments, at least two treatment sessions are performed each week, or in most of the weeks.

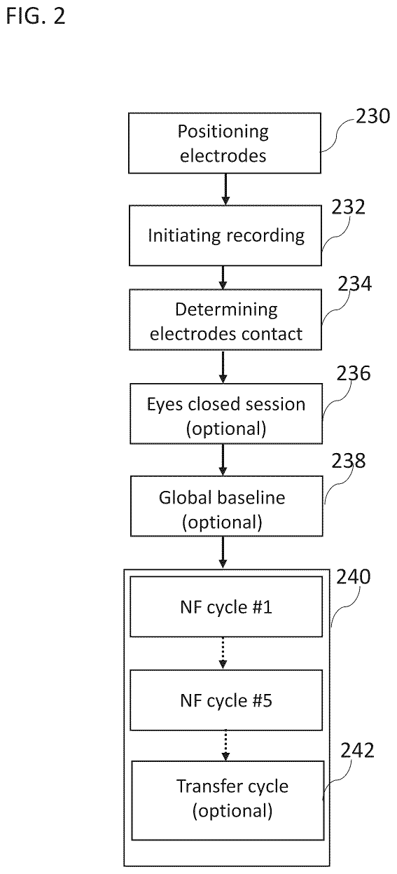

Reference is now made to FIG. 2, depicting a treatment session of a NF treatment for treating subjects diagnosed with PTSD, according to some exemplary embodiments of the invention.

According to some exemplary embodiments, one or more electrodes are positioned on a head of the patient, at block 230. In some embodiments, the one or more electrodes comprise a plurality of electrodes positioned at different locations on a head of the subject, for example at locations C3, C4, Cz, FCZ, P3, Pz and P4 of the 10-20 coordinate system, on a head of the subject, or at any combination of these locations. In some embodiments, the electrodes are attached to a head of the patient, for example to the skull, using gel.

According to some exemplary embodiments, recording from the electrodes is initiated at block 232. In some embodiments, the electrodes are used to record EEG signals. In some embodiments, recording is initiated at block 232 before or after electrodes positioning at block 230.

According to some exemplary embodiments, contacts of the electrodes with the head of the subject is determined at block 234. In some embodiments, the contact is determined based on the recording initiated at block 232. In some embodiments, contact of the electrodes with the head of the patient is presented to the supervisor, for example using an interface showing electrodes with proper contact and electrodes that are not properly contacting the head of the patient. In some embodiments, the system alerts the supervisor and/or the user of which electrodes are not properly contacted during treatment and optionally what action to take to remedy the problem. In some embodiments, recording is initiated at block 232 after determining electrodes contact at block 234.

According to some exemplary embodiments, a treatment session optionally includes an eyes closed session, at block 236. In some embodiments, during the eyes close session signals are recorded from the patient when the patient is in a rest mode, for example to calibrate the recording and/or an EEG measurement system.

According to some exemplary embodiments, a treatment session optionally includes a global baseline session, at block 238. In some embodiments, during the global baseline session, EEG signals are measured and are collected for a pre-determined time period necessary to determine an activity of the at least one limbic brain region or a biomarker of the activity, for example using an EFP of the limbic brain region. In some embodiments, the pre-determined time period is up to 3 minutes, for example up to 2.5 minutes, up to 2 minutes, up to 1.5 minutes, up to 1 minute.

Optionally, a local baseline is calculated before every NF cycle. In some embodiments, during the local baseline session, EEG signals are measured and are collected for a pre-determined time period necessary to determine an activity of the at least one limbic brain region, for example using an EFP of the limbic brain region. In some embodiments, the pre-determined time period shorter than 2 minutes, for example shorter than 1.5 minute, shorter than 1 minute, or any intermediate, smaller or larger value.

According to some exemplary embodiments, a treatment session comprises one or more training cycles, at block 240. In some embodiments, the one or more training cycles comprise 1, 2, 3, 4, 5, 6, 7 or any larger number of NF training cycles. In some embodiments, the training cycles, for example the NF training cycles are consecutive training cycles. Optionally, the NF training cycles are continuously performed at block 240. Optionally, a delay between two consecutive NF training cycles is shorter than 2 minutes, for example shorter than 60 seconds, shorter than 30 seconds, shorter than 10 seconds, shorter than 5 seconds, shorter than 1 second or any intermediate, shorter or longer time period. Optionally, the training cycles comprise at least one transfer cycle 242, in which feedback regarding the activation level of the at least one limbic brain region was not delivered to the patient.

According to some exemplary embodiments, during the NF cycles, the patient watches a patient interface, for example a visual, an audio, a tactile and/or an olfactory interface which presents a scenario that changes according to the determined activity level of the at least one limbic brain region, while performing a task, for example a cognitive task and/or an emotional task. In some embodiments, the cognitive task is selected to increase the activity level of the at least one limbic brain region or a biomarker thereof. In some embodiments, an increase of the activity of the at least one limbic brain region or the biomarker thereof, increases or decreases the amount of stimulating cues delivered to the patient as part of the interface. Additionally, human detectable indications, for example visual, audio, tactile, and/or olfactory indications delivered to the subject while watching the interface indicate if a target activity level of the limbic brain region or biomarker thereof was crossed or if the determined activity is closer or is in a desired direction towards the target activity level.

### Exemplary System for Delivery of NF Treatment

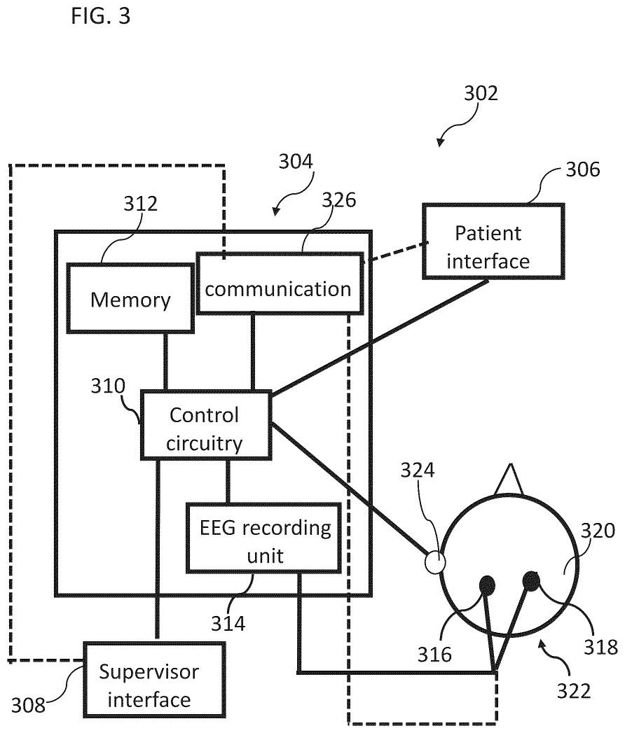

Reference is now made to FIG. 3, depicting a system for delivery of a NF treatment, according to some exemplary embodiments of the invention.

According to some exemplary embodiments, a system 302 comprises a control unit 304, a patient interface 306 and optionally a supervisor interface 308. In some embodiments, the patient interface 306 and the supervisor interface are functionally coupled to a control circuitry 310 of the control unit 304. In some embodiments, the control unit 304 further comprises a memory 312 which stores at least one algorithm and/or a lookup table, to be used when determining an activity level of at least one limbic brain region. Alternatively or additionally, the memory 312 comprises at least one EFP, for example an EFP model of the at least one limbic brain region, for example the EFP model described in for example as described in international patent application publication no. WO2012104853A2, filed on 2 Feb. 2012, and as described in international patent application publication no. WO2020121299A1, filed on 9 Dec. 2019, the contents of which is incorporated as a reference in their entirety. In some embodiments, the EFP model comprises an EFP model of the Amygdala. In some embodiments, the EFP model is applied on at least one EEG signals measured from electrodes on a head of the subject in order to determine an activity of the Amygdala. In some embodiments, the memory stores at least one NF treatment protocol or a plurality of NF treatment protocols.

According to some exemplary embodiments, the control unit 304 comprises an EEG recording unit 314 functionally coupled to the control circuitry 310. In some embodiments, the EEG recording unit 314 delivers signals from one or more electrodes positioned on a head of a subject to the control circuitry 310 for processing of the received signals and analysis of the processed signals in order to determine an activity level of the at least one limbic brain region, and/or a relation between the determined activity and at least one reference value or indication thereof, stored in the memory 312. In some embodiments, the control circuitry 310 determines the activity level or the relation between the activity level and the at least one reference value or indication thereof, using the at least one of an algorithm, a lookup table and an EFP stored in the memory 312, for example as described in international patent application publication no. WO2012104853A2, filed on 2 Feb. 2012, and as described in international patent application publication no. WO2020121299A1, filed on 9 Dec. 2019, the contents of which is incorporated as a reference in their entirety.

According to some exemplary embodiments, the EEG recording unit is functionally coupled to at least one electrode, for example a plurality of electrodes 316 and 318 positioned on a head 320 of a patient 322. In some embodiments, the plurality of electrodes comprise 2,3,4,5,6,7,8,9,10 or any larger number of electrodes positioned and optionally attached to the head 320 of the patient 322. Optionally, the plurality of electrodes are arranged in an array. In some embodiments, the electrodes are positioned at specific locations on a head of the subject, for example at locations C3, C4, Cz, FCZ, P3, Pz and P4 of the extended 10-20 coordinate system, or any combination of the locations. Alternatively, electrodes are positioned in any location or combination of locations of the extended 10-20 coordinate system. In some embodiments, the control unit 304 is connectable to at least one speaker or earphone 324 configured to deliver an audio signal to the patient 322.

According to some exemplary embodiments, the control unit comprises a communication circuitry 326 configured to receive and/or deliver signals, for example wireless signals to a remote device located outside the supervisor clinic, for example to a remote computer, a remote server, a remote cloud, a remote storage cloud, a remote processing cloud. In some embodiments, the remote device stores the at least one algorithm, lookup table and/or the EFP. In some embodiments, the remote device stores a plurality of NF treatment protocols. In some embodiments, the control unit 304 transmits the electrical signals or processed electrical signals to the remote device via the communication circuitry 326 and receives via the communication circuitry 326 signals which indicate an activity level of the limbic brain region and/or a relation between the activity level of the limbic brain region and a reference value indicating a target activation level of the limbic brain region.

According to some exemplary embodiments, the control circuitry 310 signals the patient interface 306 to display on a screen a visual interface and/or an audio interface and/or tactile and/or olfactory which continuously changes in response to an activation level or changes in the activation level relative to a baseline, of the limbic brain region or of a biomarker thereof. In some embodiments, the interface is updated every 0.5 second, every 1 second, every 5 seconds, every 10 seconds, every 15 seconds, every 20 seconds, every 25 seconds or any intermediate, smaller or larger value, based on the signals received form the electrodes, for example electrodes 316 and 318. In some embodiments, the patient interface 306 changes the visual interface presented to the patient in a delay of about 5 seconds, about 10 seconds, about 15 seconds, about 20 seconds, about 25 seconds, or any intermediate, smaller or larger value, relative to the timing in which the electrical signal is received from the electrodes 316 and 318. In some embodiments, the patient interface comprises a display and/or speakers.

According to some exemplary embodiments, the patient interface, for example a visual interface, is delivered in two dimension (2D), or in three dimension (3D). In some embodiments, the patient interface is delivered using virtual reality, augmented reality, with or without at least one of, audio, olfactory and tactile feedback.

According to some exemplary embodiments, the control circuitry 310 signals the supervisor interface to deliver visual and/or audio indications to a supervisor of the treatment. In some embodiments, the visual and/or audio indications present at least one of, a progress of the patient during the treatment, activity level of the limbic brain region or biomarker thereof, for example the Amygdala, changes in the activity level of the limbic brain region, difference between a reference value and an activity level of the limbic brain region, stage of the NF session, actions of the system and indications or cues delivered to the patient. Optionally, the supervisor interface 308 is a remote device of the supervisor coupled to the control unit 304 via the communication circuitry 326. In some embodiments, the control unit 304 transmits at least one of, data collected from the patient, data transmitted or displayed to the supervisor, to the remote device, for example for generating a database. In some embodiments, the dataset includes information collected from a plurality of systems and/or from a plurality of patients.

According to some exemplary embodiments, at least one of, the patient interface 306, the electrodes 316 and 318, and the supervisor interface 308 is in communication, for example using wireless communication and wireless signals, with the communication circuitry 326.

According to some exemplary embodiments, the control circuitry 310 is configured to deliver a NF treatment to a PTSD patient based on at least one protocol or indications thereof stored in the memory 312. Additionally, the control circuitry 310 is configured to signal at least user interface, for example the supervisor interface 318 to generate an indication with information to perform an assessment of results of the NF treatment within a time period between 8 weeks and 12 weeks from initiating the delivery of the NF treatment.

According to some exemplary embodiments, the control circuitry 310 is configured to receive an input signal with information regarding results of the assessment and to signal the at least one user interface to generate an indication with information which includes a suggestion to modify the at least one NF treatment protocol or to stop the delivery of the NF treatment, according to the results of the assessment.

According to some exemplary embodiments, the memory 312 stores a plurality of protocols of a NF treatment. In some embodiments, the control circuitry 310 is configured to receive an input signal with information regarding results of the assessment and to signal the at least one user interface to generate an indication with information which includes a suggestion to replace the at least one protocol of the NF treatment with a different protocol of the plurality of protocols, according to the results of the assessment.

According to some exemplary embodiments, the system comprises at least one remote device which stores a plurality of protocols of the NF treatment. In some embodiments, the communication circuitry 326 is configured to communicate with the at least one remote device. In some embodiments, the control circuitry 310 is configured to receive an input signal with information regarding results of the assessment and to signal the at least one user interface to generate an indication with information which includes a suggestion to replace the at least one protocol of said NF treatment with a different protocol of the plurality of protocols stored in the remote device, according to the assessment results.

According to some exemplary embodiments, the control circuitry 310 is configured to signal the patient interface 306 to present an interface stored in the memory 312 to the patient 322, for example a PTSD patient. Additionally, the control circuitry 310 is configured to measure EEG signals using the electrical signals received by said EEG recording unit 314. In some embodiments, the control circuitry 310 detects a change in activity of the Amygdala or in activity of at least one related brain region using the measured EEG signals and at least one algorithm and/or an electrical fingerprint (EFP) of the Amygdala or of at least one related brain region stored in the memory 312. In some embodiments, the control circuitry 310 modifies the presentation of the interface to patient 322 according to the detected change in activity.

### Exemplary PTSD Assessment

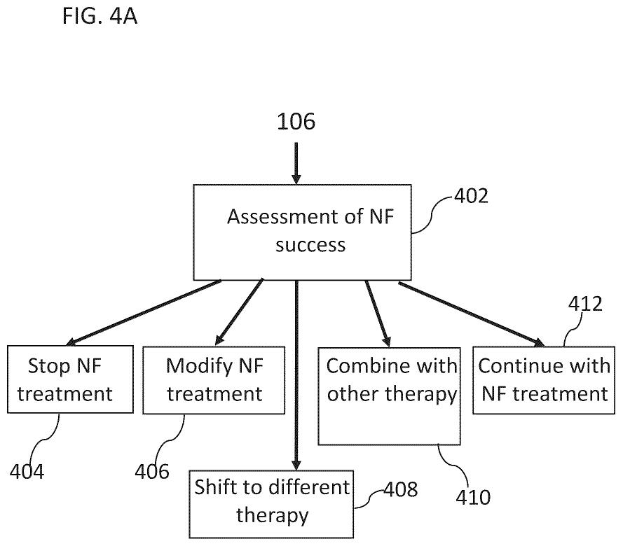

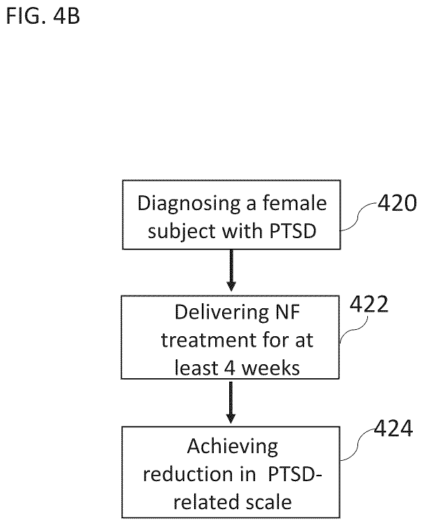

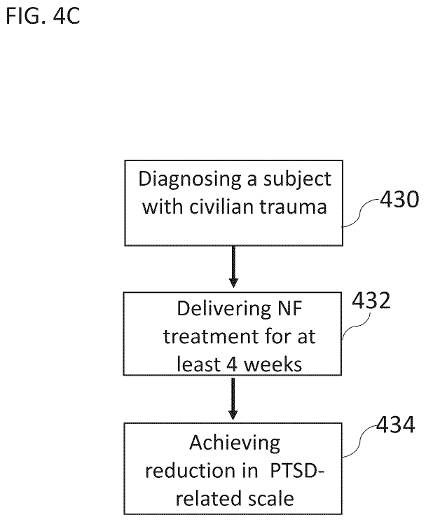

Reference is now made to FIG. 4A, depicting assessment of a NF treatment, according to some exemplary embodiments of the invention.

According to some exemplary embodiments, a NF treatment is delivered to a subject for several weeks, for example as described at block 106 shown in FIG. 1. In some embodiments, the NF treatment is delivered during a time period of at least 8 consecutive weeks. In some embodiments, the NF treatment is delivered during a time period between 8 consecutive weeks and 12 consecutive weeks. In some embodiments, NF treatment is delivered during a time period of non-consecutive weeks.

According to some exemplary embodiments, assessment of a success of the NF treatment is performed after 8 to 12 weeks from initiating the delivery of the NF treatment, at block 402. Optionally, the assessment is performed during the delivery of the NF treatment. In some embodiments, the assessment comprises determining a change in a score of one or more PTSD related scales following the NF treatment compared to a baseline score of the scales prior to NF treatment initiation. In some embodiments, the one or more related PTSD scales comprise at least one of, CAPS-5, PCL-5, Emotion Regulation Questionnaire (ERQ), Patient Health Questionnaire (PHQ-9) and Clinical Global Impression (CGI). Alternatively or additionally, the assessment is performed by a healthcare professional not using any of the above scales.

According to some exemplary embodiments, the NF treatment is stopped, at block 404. In some embodiments, the NF treatment is stopped based on the results of the assessment performed at block 404. In some embodiments, the NF treatment is stopped, for example if the assessment indicates that there is no improvement in a state of a subject undergoing the NF treatment or there is a deterioration in a state of the subject.

According to some exemplary embodiments, the NF treatment is modified at block 406. In some embodiments, at least one parameter of the NF treatment is modified at block 406 based on the assessment results. In some embodiments, the at least one parameter comprises at least one of, number of treatment sessions, number of weeks for the treatment, length of each treatment session, number of sessions per week, interval between two sessions. In some embodiments, the modified NF treatment is delivered to the subject.

According to some exemplary embodiments, the subject is shifted to a different therapy, for example a different mental therapy, at block 408. In some embodiments, the subject is shifted to a different therapy based on the assessment results. In some embodiments, the subject is shifted to a different therapy for example if the assessment indicates that there is no improvement in a state of a subject undergoing the NF treatment or there is a deterioration in a state of the subject. In some embodiments, the different mental therapy comprises at least one of, psychological therapy, psychiatric therapy, and therapy using a bioactive agent, for example a drug.

According to some exemplary embodiments, the NF treatment is combined with additional therapy at block 410. In some embodiments, the NF treatment is combined with additional therapy based on the assessment results. In some embodiments, the additional therapy comprises at least one of, an additional NF treatment directed to activity modulation of a different brain region or a biomarker thereof, psychological therapy, psychiatric therapy, and therapy using a bioactive agent, for example a drug.

According to some exemplary embodiments, the NF treatment is continued at block 412. In some embodiments, the NF treatment delivered at block 106 is continued based on the assessment results. In some embodiments, the NF treatment is continued if the assessment results indicate a projected success in achieving one or more NF treatment goals if the NF treatment is continued.

### Exemplary NF Treatment of Specific Sub-Populations

According to some exemplary embodiments, the NF treatment is delivered to subjects of specific sub-populations that are more suitable to receive the NF treatment compared to the entire population.

Reference is now made to FIG. 4B, depicting delivery of the NF treatment to female subjects diagnosed with PTSD, according to some exemplary embodiments of the invention.

According to some exemplary embodiments, a female subject is diagnosed with PTSD at block 420, for example as described at block 102 of FIG. 1.

According to some exemplary embodiments, NF treatment is delivered to the female subject for at least 8 weeks, for example for at least 10 weeks, optionally consecutive weeks, optionally consecutive weeks, at block 422. In some embodiments, the NF treatment is delivered for a time period between 4 consecutive weeks and 12 consecutive weeks, for example between 8 consecutive weeks and 12 consecutive weeks.

According to some exemplary embodiments, a reduction in a PTSD scale or a comparable metric is achieved at block 424, following the NF treatment.

Reference is now made to FIG. 4C, depicting delivery of the NF treatment to subjects diagnosed with PTSD originating from non-non-civilian trauma, according to some exemplary embodiments of the invention.

According to some exemplary embodiments, a subject having PTSD originating from non-non-civilian trauma is diagnosed with PTSD at block 430, for example as described at block 102 of FIG. 1.

According to some exemplary embodiments, NF treatment is delivered to the female subject for at least 4 consecutive weeks, for example for at least 8 consecutive weeks, at block 432. In some embodiments, the NF treatment is delivered for a time period between 4 consecutive weeks and 12 consecutive weeks, for example between 8 consecutive weeks and 12 consecutive weeks.

According to some exemplary embodiments, a reduction in a PTSD scale is achieved at block 434, following the NF treatment.

### Exemplary System and Method for Assessment of a PTSD Treatment

According to some exemplary embodiments, assessment of an effect of a PTSD treatment performed on a subject, for example a patient, or a group of patients, is performed by a device, for example an assessment device. In some embodiments, the device receives information which includes results of at least one assessment of a subject, for example a cognitive, mental and/or functional assessment, performed after the subject receives a NF treatment for at least one week. In some embodiments, the information includes results of a subject assessment performed after the subject receives a NF treatment for at least 4 weeks, after at least 8 weeks or any intermediate, smaller or larger number of weeks. In some embodiments, the information includes results of a subject assessment performed after the subject receives at least one session of NF treatment or after at least one week of NF treatment.

According to some exemplary embodiments, the information is used to determine whether the given NF treatment advances according to an expected improvement path, or whether the NF treatment is not effective enough for the specific subject or for a population. In some embodiments, if the system determines that the NF treatment delivered to the patient or to a group of patients is not effective enough, the system provides indication with instructions, for example suggestions or recommendations, how to modify the NF treatment, or to replace or combine the NF treatment with a different treatment, or to stop the NF treatment.

According to some exemplary embodiments, the assessment is performed during a NF treatment plan of a subject, for example to increase an effect of the NF treatment or an overall treatment on the subject. Alternatively or additionally, the assessment is performed in order to provide a more efficacious treatment to at least one new subject starting a new treatment plan.

According to some exemplary embodiments, the assessment allows to identify at least one sub-population in a larger population of subjects, in which the NF treatment is less effective or more effective compared to the effect in a larger population, overall population, or compared to a different sub-population, and to modify the NF treatment or an overall treatment provided to a subject which belongs to the at least one sub-population, to optionally increase the effect of the treatment delivered to the subject.

According to some exemplary embodiments, the assessment allows to define at least one new sub population or a plurality of sub populations whose response differs from the response of other identified sub populations and/or response of a larger sub population.

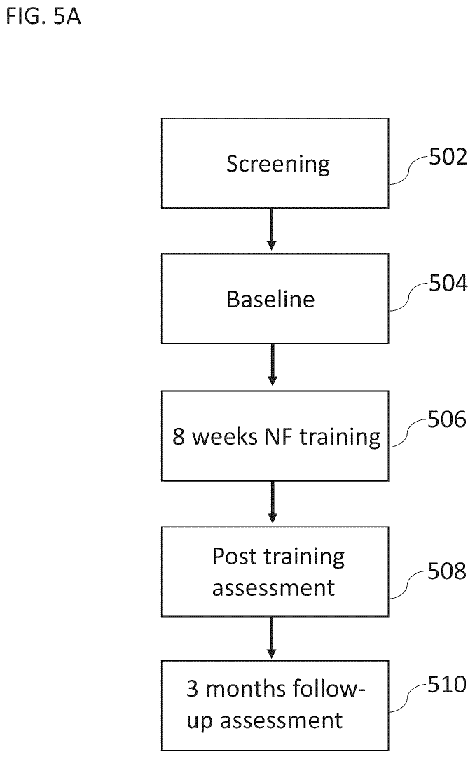

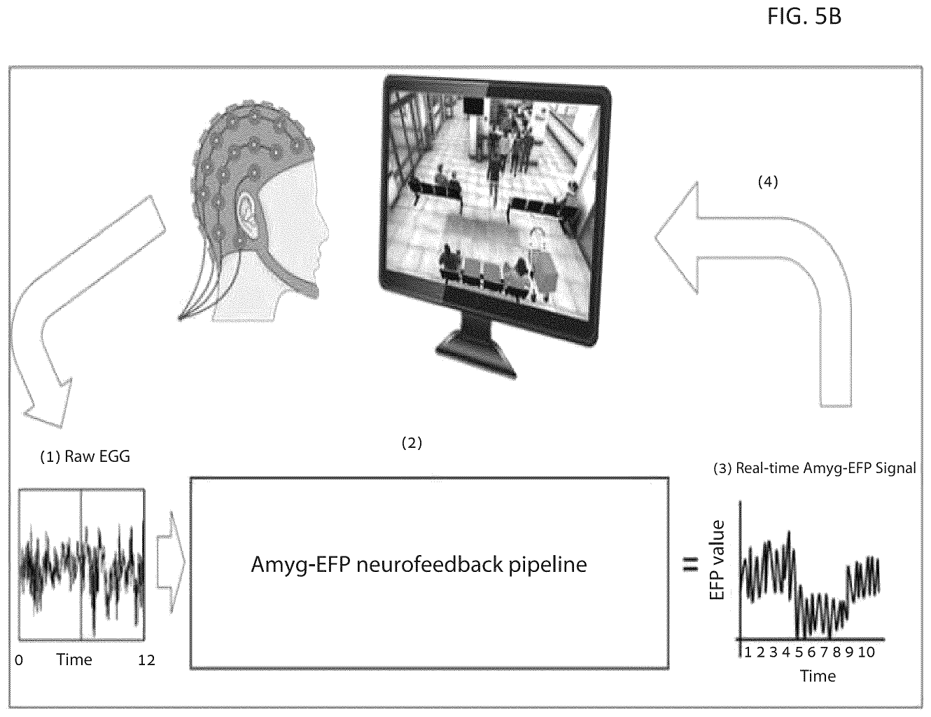

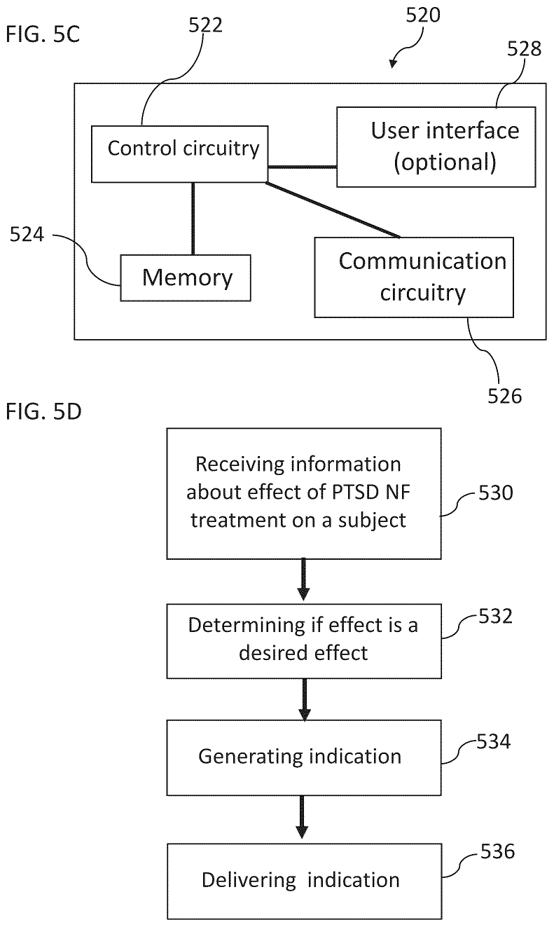

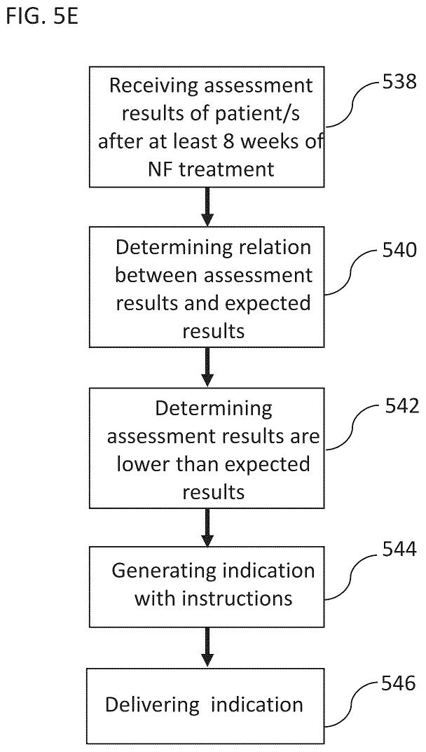

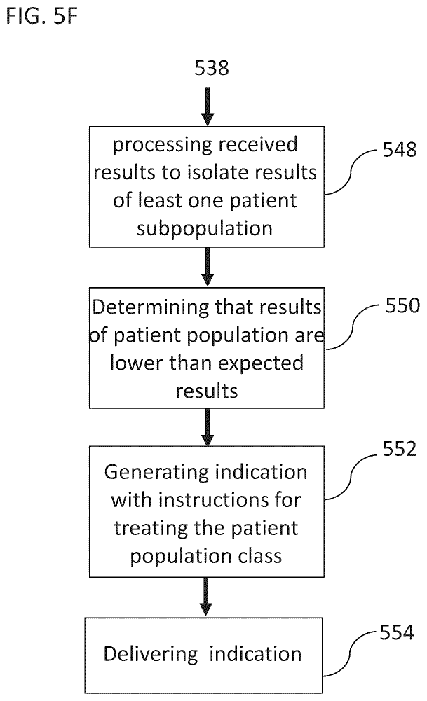

Reference is now made to FIG. 5C, depicting a device for assessment of an effect of a NF treatment, according to some exemplary embodiments of the invention.

According to some exemplary embodiments, the device 520, for example the assessment device is a standalone device used for monitoring of a NF PTSD treatment and/or for management of PTSD patients. Alternatively or additionally, the assessment device is a component of a device or a system for delivery of the NF treatment. In some embodiments, the NF treatment is a NF treatment which trains a patient to control, for example reduce activity of a limbic brain region or limbic network, for example reduce activity of the Amygdala. In some embodiments, the NF treatment is based only on EEG recordings and a model, for example a fingerprint, used for interpretation of the recorded EEG signals into indications of activity or modulation of the activity, of at least one of, the limbic brain region, the limbic network, and/or the amygdala.

According to some exemplary embodiments, the device 520 is a server, a cloud storage and/or processing, or any remote device that is in communication directly or indirectly with the patient and/or with an expert, for example a therapist, a physician, or a health care personnel delivering and/or monitoring the NF treatment.

According to some exemplary embodiments, the device comprises a control circuitry 522, for example a controller, and a memory circuitry, for example memory 524. In some embodiments, the memory 524 stores information of at least one patient for example a patient diagnosed with a stress disorder, for example PTSD. Additionally or alternatively, the memory 524 stores information which includes results or indications thereof, of at least one patient assessment, performed after at least one session, or after at least one week, for example after at least 4 weeks, after at least 8 weeks, or any intermediate, shorter or longer time period from initiating the delivery of the NF treatment to the patient or to a group of patients.

According to some exemplary embodiments, the results are results of a patient assessment performed using at least one assessment scale, for example CAPS-5 scale, CGI scale, PCL-5 scale, ERQ scale, and/or a PHQ-9 scale. In some embodiments, the results comprise at least one central tendency measure of the results, for example mean, median and mode, of the assessment results.

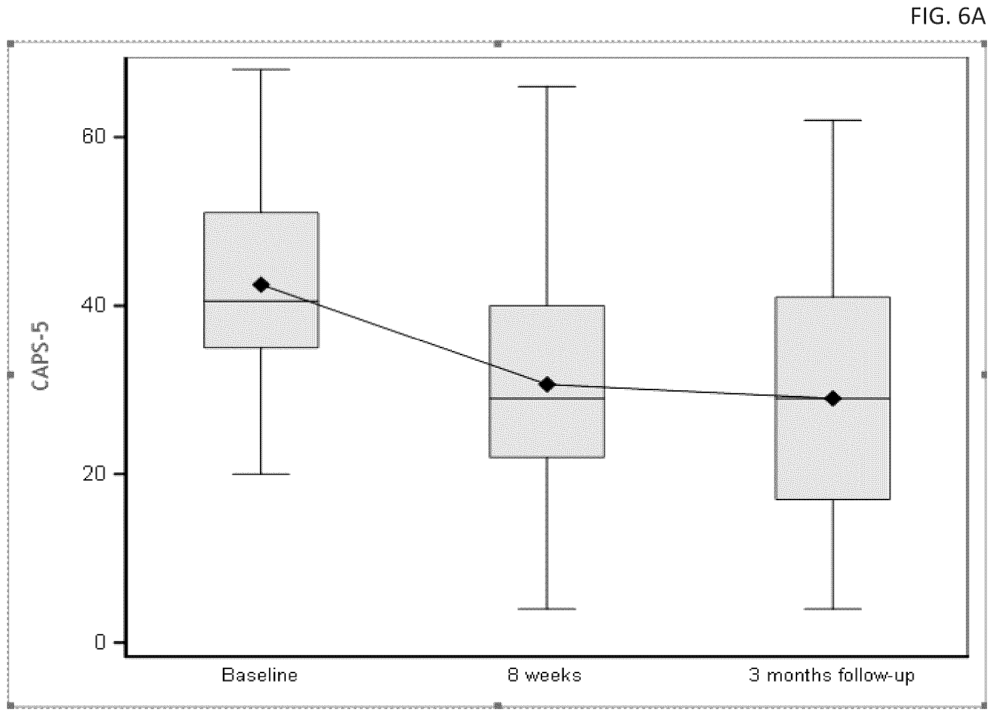

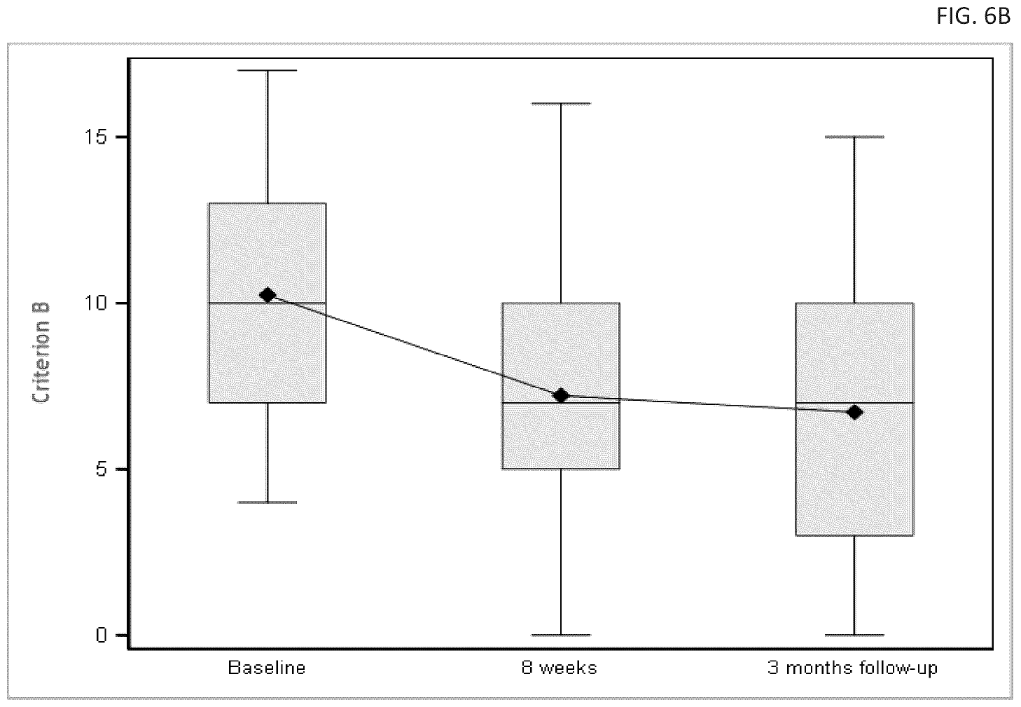

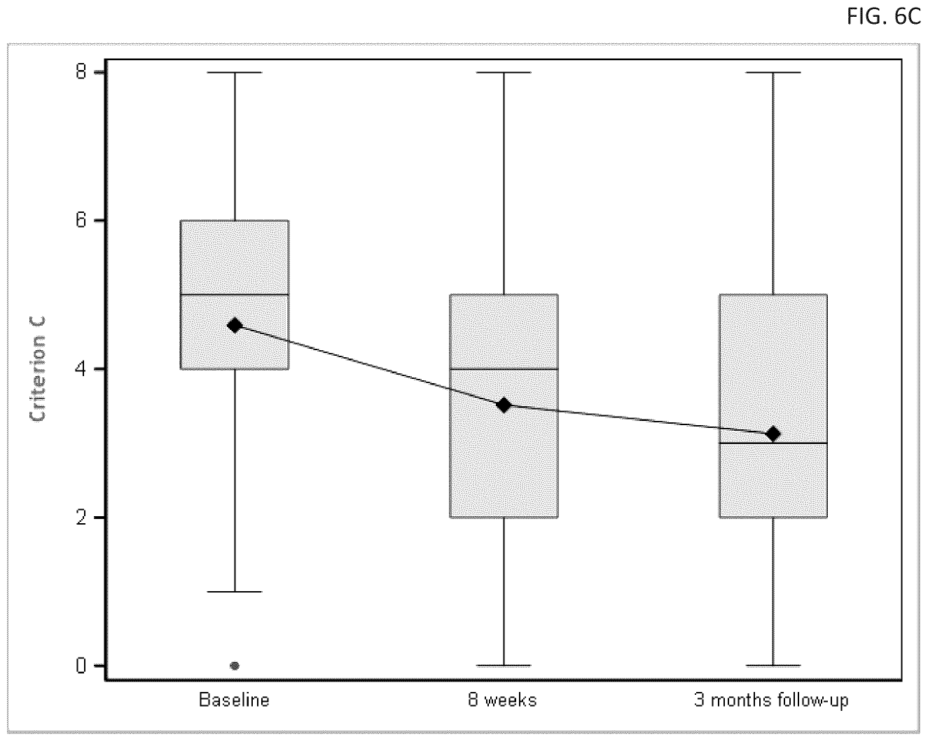

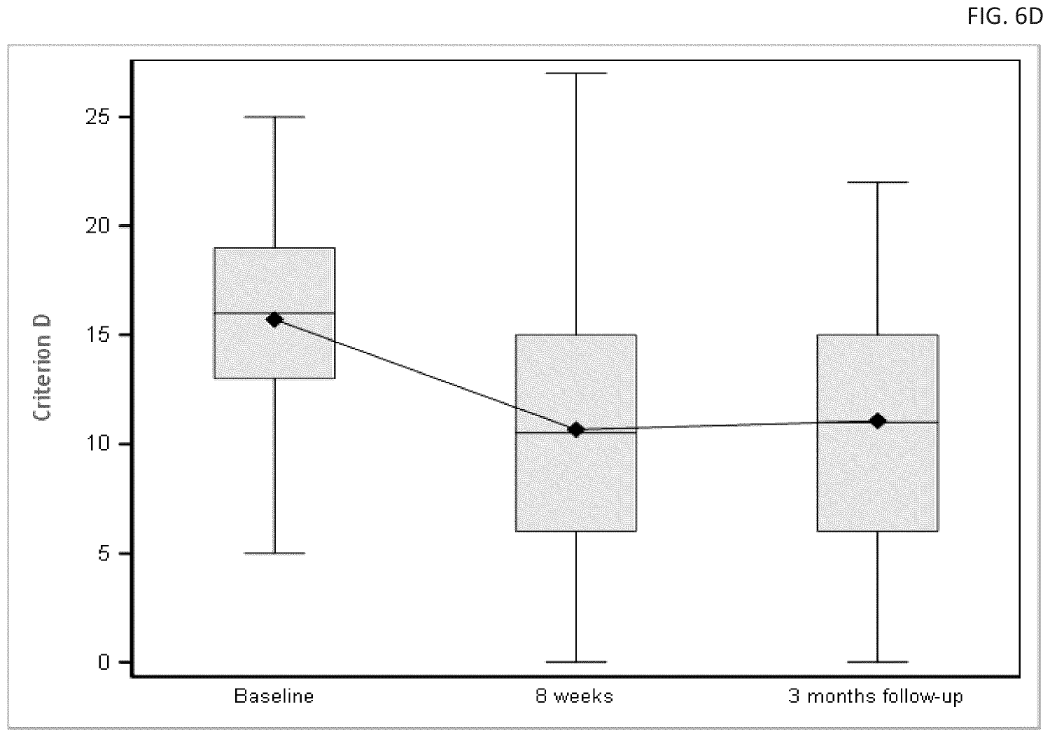

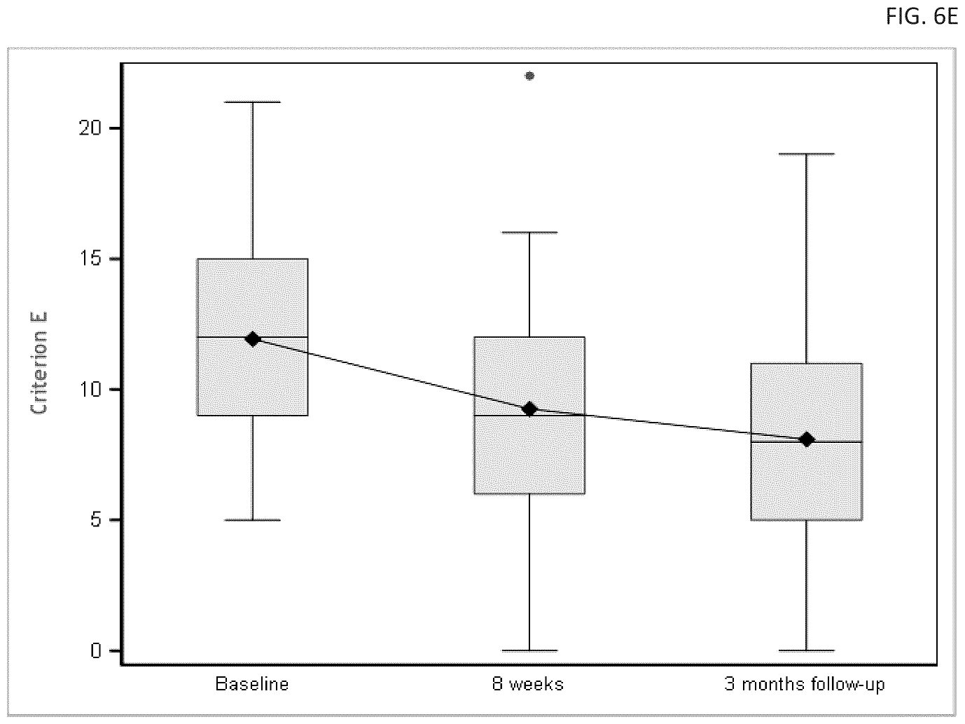

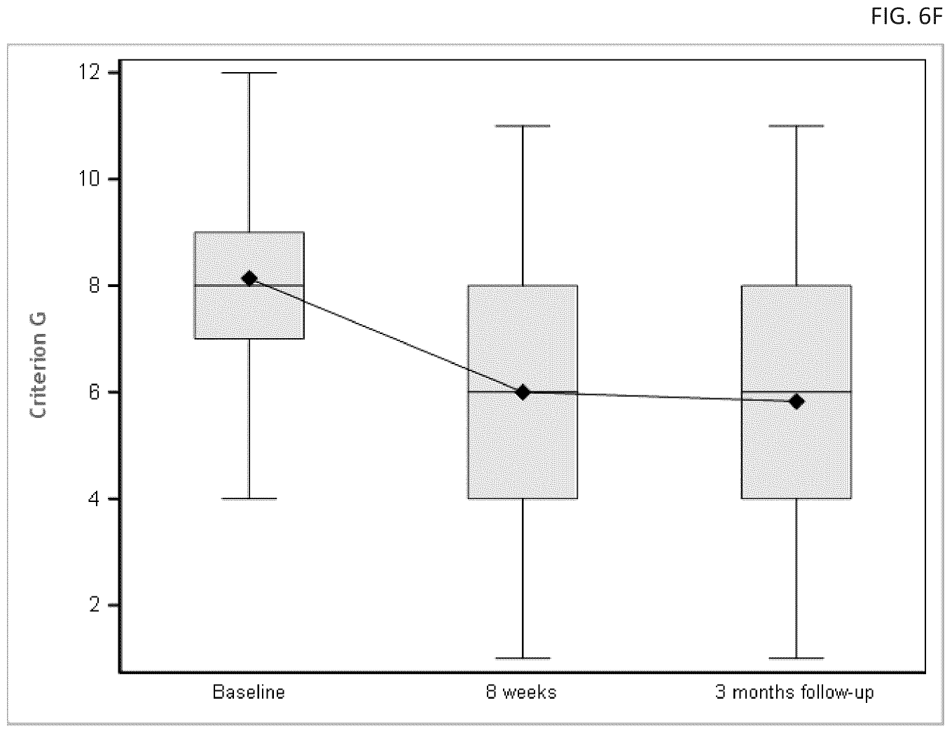

Additionally, the memory 524 comprises expected measurements of the at least one assessment, or indications thereof. In some embodiments, the expected results are in a form of a formula, model or algorithm. Optionally, the expected measurements comprise a central tendency measure of the expected measurements. In some embodiments, the expected measurements are based on the assessment results presented in Table A, and/or on at least one graph of the graphs shown in FIGS. 6A-6F, 7A-7F, 8A-8B, and 9A-9B.

According to some exemplary embodiments, the memory 524 stores one or more algorithm, formula, model and/or lookup table for processing and/or analyzing the patient assessment results and the expected results and optionally for determining a relation between the assessment results and the expected results. In some embodiments, one or more algorithm, formula, model and/or lookup table stored in the memory are used to generate at least one indication optionally with instructions to a user or an expert, based on at least one of, the assessment results, the expected results and/or the determined relation.

According to some exemplary embodiments, the control circuitry 522 is configured to at least one of, process the received assessment results, process the stored expected results, determine a relation between the received assessment results and the expected results, determine that the received assessment results are lower than the expected results and to generate at least one indication based on the determined relation, using any content, for example any information, stored in the memory 524.

According to some exemplary embodiments, the control circuitry 522 is configured to generate at least one indication, for example a human detectable indication. In some embodiments, the control circuitry generates the indication based on the results of the processing of the assessment results and/or the processing of the expected results, and/or based on a determined relation between the assessment results and the expected results. In some embodiments, the indication comprises one or more instructions, stored in the memory 524.

According to some exemplary embodiments, the device 520 comprises a communication circuitry 526. In some embodiments, the communication circuitry 526 is configured to receive and/or transmit signals, for example wireless signals, for example radio signals, WI-FI signals, Bluetooth signals, IR signals, light signals, or any other type of signals used for wireless communications. In some embodiments, the device 520 receives the information stored in the memory 524 from a remote device, for example a device located at a distance larger than 1 meter from the device 520 or any device in communication with the device 520, via the communication circuitry 526. In some embodiments, the device 520 transmits information and/or at least one indication generated by the control circuitry 522 to a remote device, via the communication circuitry.

According to some exemplary embodiments, the device 520 optionally comprises a user interface 528. In some embodiments, the user interface 528 is used to receive input and/or to deliver at least one indication, to a user of the device 520.

Reference is now made to FIG. 5D, depicting a general process for assessment of an effect of a NF treatment on a subject as performed by a device, for example device 520, according to some exemplary embodiments of the invention.

According to some exemplary embodiments, a device, for example a device for assessment of an effect of a NF treatment, receives information about the effect of the NF treatment on at least one subject, for example a patient. In some embodiments, the information comprises results of an assessment performed before treatment and/or after initating the NF treatment, for example after at least one week, for example at least 4 weeks, at least 8 weeks, from initiating the NF treatment delivered to the patient. In some embodiments, the information comprises results of an assessment performed after at least one session of the NF treatment. In some embodiments, the assessment results comprise score of an assessment scale, for example CAPS-5 scale, CGI scale, PCL-5 scale, ERQ scale, and/or a PHQ-9 scale. In some embodiments, the received information is stored in a memory of the device, for example memory 524 shown in FIG. 5C.

According to some exemplary embodiments, the device, for example a control circuitry of the device determines if an effect of the NF treatment is a desired effect, for example a target effect, at block 532. In some embodiments, the control circuitry, for example control circuitry 522 uses at least one of, an algorithm, a formula, a model and/or a lookup table stored in the memory 524, for the determining. For example, by determining a relation between the assessment results and expected results, stored in the memory 524. In some embodiments, determining a relation comprises comparing assessment results or indications thereof, to the expected results or indications thereof. Optionally, determining a relation comprises determining a relation between a central tendency measure of the assessment results and a central tendency measure of the expected results.

According to some exemplary embodiments, the device generates at least one indication, at block 534. In some embodiments, the device 520, for example the control circuitry 522, generates the at least one indication based on the determining results, for example if the effect is a desired effect, and/or if the effect is not a desired effect. In some embodiments, the control circuitry 522 generates the indication if the assessment results are lower than the expected results, optionally indicating that the effect of the NF treatment on a subject or a group of subjects is lower or higher than an expected effect. In some embodiments, an expected effect is an effect of the NF treatment measured in a population of subjects after a specific time period from initiating the NF treatment.

According to some exemplary embodiments, the indication is a human detectable indication, for example an audio and/or a visual indication. In some embodiments, the indication includes instructions to an expert monitoring the NF treatment given to the subject, how to change the NF treatment or an overall treatment provided to the subject in view of the assessment results. For example, the indication includes instructions to stop the NF treatment, to modify at least one parameter of the NF treatment, to replace the NF treatment protocol with a different treatment protocol, and/or to replace or combine the NF treatment with at least one additional treatment or stop another treatment or modify it.

According to some exemplary embodiments, the indication is optionally delivered to a remote device, at block 536. In some embodiments, the indication is delivered to the remote device by the device 520 using the communication circuitry 526.

Alternatively or additionally, the indication, and/or the information stored in the memory 524, is used to improve at least one existing NF treatment protocol. In some embodiments, the at least one existing NF treatment protocol is stored in the memory 524 of the device 520, or in a memory of a remote device, for example a server, a cloud storage, or in a database in the remote device.

Reference is now made to FIG. 5E depicting a detailed assessment process, according to some exemplary embodiments of the invention.

According to some exemplary embodiments, an assessment device, for example device 520 shown in FIG. 5C, receives results of an assessment of a subject conducted after initiating a NF treatment, for example after at least one week from initiating a NF treatment or after at least one session of the NF treatment, at block 538. In some embodiments, the results are results of an assessment of a subject conducted at least 8 weeks from initiating a NF treatment, at block 538. In some embodiments, the results comprise results of at least one of, CAPS-5 assessment scale, CGI assessment scale, PCL-5 assessment scale, ERQ assessment scale, and/or a PHQ-9 assessment scale. In some embodiments, the subject, for example a patient diagnosed with a stress disorder, for example with PTSD, undergoes the assessment at least 8 weeks from initiating the NF treatment. Optionally, the subject undergoes the assessment at an end of a planned NF treatment, for example after finishing the last NF treatment session, or at least 1 day after completing the NF treatment or the last NF treatment session.

According to some exemplary embodiments, the assessment results are received from a remote device via a communication circuitry, for example communication circuitry 526 shown in FIG. 5C. Alternatively or additionally, the assessment results are received via a user interface of the device, for example user interface 528.

According to some exemplary embodiments, the assessment results are results of at least one assessment performed to a patient or a group of patients.

According to some exemplary embodiments, a relation between the assessment results, for example the received assessment results, and expected results, is determined at block 540. In some embodiments, the expected results or indications thereof are stored in a memory of the device in a form of at least one central tendency of the expected results, a formula, a model, an algorithm or a lookup table. In some embodiments, the expected results are results of the at least one assessment measured after a specific time period similar to the time period in which the received assessment results were measured, in subjects where the NF treatment had a desired effect, for example a target effect, on the subjects.

According to some exemplary embodiments, the device determines that the received assessment results are lower than the expected results, at block 542. In some embodiments, determining that the received assessment results are lower than expected results indicate that the effect of the NF treatment on the subject is lower than a target desired effect of the NF treatment or greater than an expected effect.

Alternatively, the device determines that the received assessment results are higher than the expected results, indicating that the effect of the NF treatment is larger than an expected effect.

According to some exemplary embodiments, in case the assessment comprises CAPS-5, the expected results comprise a reduction of at least 6 points in the CAPS-5 scale after at least 4 weeks, for example after at least 4 weeks of NF treatment or after finishing at least 50% of planned NF treatment sessions.

According to some exemplary embodiments, in case the assessment comprises PCL-5, the expected results comprise a reduction of at least 9 points in the PCL-5 scale after at least 4 weeks, for example after at least 8 weeks of NF treatment, or after finishing at least 50% of planned NF treatment sessions.

According to some exemplary embodiments, the device generates at least one indication, at block 544. In some embodiments, the device 520, for example the control circuitry 522 generates the indication based on the determined relation, or based on the determining that the assessment results are lower than the expected results or higher. In some embodiments, the indication comprises instructions how to approve the treatment given to the patient, for example a NF treatment or a combination of the NF treatment with at least one additional treatment, to increase the effect of the NF treatment on the subject. In some embodiments, the assessment results measure a state of the patient following the NF treatment and optionally during the NF treatment, for example a clinical state, a mental state, a cognitive state and/or a functional state of the patient. In some embodiments, the indication comprises instructions how to improve the state of the subject.

According to some exemplary embodiments, the instructions include instructions to modify at least one parameter of an existing NF treatment delivered to the patient, and optionally to deliver at least one NF treatment session to the patient with the modified parameter. In some embodiments, the at least one parameter comprises at least one of, duration of a NF treatment session, number of additional NF treatment sessions, type and/or content of a visual interface presented to the patient during a NF treatment session, analysis performed to EEG signals recorded from the subject brain during a NF treatment session.

According to some exemplary embodiments, the instructions include instructions to replace an existing NF treatment protocol with a different protocol, and optionally to deliver at least one treatment session to the patient using the different protocol.

According to some exemplary embodiments, the instructions include stopping, optionally temporarily, the NF treatment delivered to the subject. Alternatively, the instructions include instructions to deliver to the patient at least one additional NF treatment session using the existing NF treatment protocol.

According to some exemplary embodiments, the instructions include replacing the NF treatment with at least one different treatment. Alternatively, the instructions include combining the NF treatment using the existing or a different treatment protocol, with at least one different treatment. In some embodiments, the at least one different treatment comprises at least one of, a cognitive behavioral treatment (CBT), a psychological treatment, a psychiatric treatment, and/or a drug treatment.

According to some exemplary embodiments, in case the received assessment results indicate that the effect of the NF treatment is larger than an expected effect, the generated indication includes instructions to modify the NF treatment or an overall treatment delivered to the subject. For example, the indication includes instructions to shorten the NF treatment, to shorten a NF treatment session, to increase a difficulty level of at least one element of the NF treatment session, ot increase a time between NF treatment sessions and/or to stop the NF treatment.

According to some exemplary embodiments, the generated indication is delivered, at block 546. In some embodiments, delivering of the generated indication comprises transmitting the indication to a remote device, for example to a remote computer, using the communication circuitry 526.

According to some exemplary embodiments, the received results include assessment results of a large population of patients, for example a population of at least 10 patients. In some embodiments, the device is configured to process the received results, for example by sorting or classifying the received results according to a specific sub-population of patients that performed the assessment, for example a population of female subjects, a population of male subjects, a population of subjects in which the PTSD originated from a civilian trauma, and a population of subjects in which the PTSD originated from a non-civilian trauma.

According to some exemplary embodiments, the device uses the classified results of the sub-population to determine an effect of the NF treatment on the specific sub-population, and to modify the treatment provided to the sub-population if the effect of the NF treatment is lower than an expected target effect for this sub-population.

Reference is now made to FIG. 5F, depicting a process for determining an effect of NF treatment on specific sub-populations of patients, according to some exemplary embodiments of the invention.

According to some exemplary embodiments, the assessment results received at block 538 comprise assessment results from a group of patients. In some embodiments, each patient performed the at least one assessment after initiating the NF treatment, for example after at least 4 weeks of NF treatment.

According to some exemplary embodiments, the device processes the received results to isolate results of at least one specific subpopulation of patients, at block 548. In some embodiments, the processing comprises sorting or classifying the overall results, in order to isolate the results of the at least one specific subpopulation. In some embodiments, the processing further comprises calculating a central tendency of the isolated results.

According to some exemplary embodiments, the device determines that the isolated results or central tendency thereof, are lower or greater than expected results, at block 550. In some embodiments, the device determines a relation between the isolated results and the expected results, in order to determine that the isolated results are lower than the expected results. In some embodiments, the expected results are results of the at least one assessment in the subpopulation f patients which indicate a desired, for example a target effect, of the NF treatment on patients associated with the subpopulation.

According to some exemplary embodiments, an indication is generated at block 552. In some embodiments, the indication is based on the determined relation between the results of the subpopulation and the expected results. Alternatively, the indication is generated in response to the determining that the results of the subpopulation are lower relative to the expected results. In some embodiments, the generate indication includes instructions to modify the NF treatment delivered to patients which belong to the at least one subpopulation, for example as described at block 544 of FIG. 5E.

According to some exemplary embodiments, the indication is delivered at block 554, for example as described at block 546 of FIG. 5E.

According to some exemplary embodiments, the processing of the received assessment results in order to identify and isolate results of a specific subpopulation, is performed after determining that the assessment results of a larger population are lower than expected results, indicating that the NF treatment has an effect which is lower than a target effect on the larger population. In some embodiments, the processing is performed in order to identify at least one specific subpopulation in which the effect of the NF treatment is lower than expected and to provide a more adapted NF treatment for this specific subpopulation.

In some embodiments of the invention, part or all of the methods described in FIGS. 5D-5F are optionally performed by a different device from the device described in FIG. 5C.

### Exemplary Clinical Study

### Clinical Study

A clinical study was performed in order to assess the clinical effect of the NF treatment on a population of human subjects diagnosed with PTSD, for example chronic PTSD.

Reference is now made to FIG. 5A, depicting a flow chart of the study protocol. Subjects were screened at block 502, for example to select the subject population for the study. In the clinical study and in some embodiments of the invention, the subject population consist of the following inclusion criteria, subjects in the age between 22 to 65, subjects diagnosed with PTSD established according to the Diagnostic and Statistical Manual of Mental Disorders, Fifth Edition (DSM-V), subjects with One (1) to twenty (20) years since index trauma, subjects. In addition, the subject population for the clinical study consist of the following additional inclusion criteria, subjects of any gender, subjects that were able to give signed, informed consent, subjects having normal or corrected-to-normal vision, subjects having normal or corrected to normal hearing, and subjects that are able to adhere to the study schedule.

During the screening at block 502, participants who agree to participate in the study and sign an informed consent form will undergo screening according to the eligibility criteria and confirmation of the diagnosis, using the International Neuropsychiatric Interview (MINI for DSM-V).

Baseline assessments of clinical outcome measures was recorded, at block 504. In addition, during the baseline or combined with the first NF treatment, for example a EEG-NF treatment, the subject went through ˜10 minutes ‘pre-training’ session to become familiar with the system prior to the actual EEG-NF treatment sessions.

NF treatment, also termed herein as NF training was performed at block 506. Each of the study subjects performed fifteen (15) treatment sessions, also termed herein as training sessions in total, delivered with a frequency of two sessions per week, on non-consecutive days, for a total treatment duration, also termed herein as training duration, of eight (8) consecutive weeks. The first training session took place no more than 7 days after the Baseline Assessment at block 504. In the study and in some embodiments of the invention, each training session lasted approximately about 30 minutes, including: EEG electrode positioning, signal quality testing, and EEG-NF training (the net duration of neurofeedback training itself is about 25 minutes per session).

FIG. 5B shows the use of the EFP in the system neurofeedback paradigm in the study and in some embodiments of the invention: the recorded EEG data (1) are cleaned and transformed as described above (2) and then multiplied by the coefficients of the EFP model's time delay×frequency×weight matrix (3) to produce the EFP value (4). Changes in the EFP values (relative to Local Baseline) are then reflected as changes in the animated audio-visual interface.

Post-training assessment was performed at block 508. In the study and in some embodiments of the invention, the first follow-up assessment was performed immediately following the last EEG-NF training session or within 7 days thereafter (8-9 weeks). All clinical outcome measures were evaluated.

A 3-months follow up assessment was performed at block 510. In the study and in some embodiments of the invention, the second follow-up assessment was performed 3 months following the completion of the last EEG-NF training session. All clinical outcome measures were be measured.

Each subject participated in the study for a total of approximately 5 months (˜22 weeks, from Screening Visit to the last (3 months) Follow-up Assessment Visit).

During the study different assessment tools, for example assessment scales were used to evaluate a state of a subject participating in the study, following the NF training compared to baseline measurements. One or more of the assessment scales are used in some embodiments of the invention. The assessment scales comprise, CAPS-5, Clinical Global Impression (CGI) scale, PTSD Checklist for DSM-5 (PCL-5), Emotion Regulation Questionnaire (ERQ), and a Patient Health Questionnaire (PHQ-9).

### Clinical Study Results

In the clinical study and in some embodiments of the invention, a state of subjects, for example PTSD patients, receiving the NF treatment is assessed following the treatment, for example following 8-12 weeks of NF treatment, and/or 3 months after completing the NF treatment.

Table A below summarizes changes in scores of different scales after 8 weeks of NF training, in the total population and in different sub-populations (compared to baseline scores):

TABLE A

| Change from | CAPS-5 | PCL-5 | PHQ-9 | ERQ |
|---|---|---|---|---|
| Baseline: Week 8 | Mean | Mean | Mean | Mean |
| Total Population | −11.8 | −9.6 | −2.9 | 0.2 |
| Females | −15.3 | −14.6 | −4.6 | 0.9 |
| Males | −8.6 | −4.8 | −1.3 | −0.4 |
| Non-civilian Trauma | −7.4 | −3.9 | −1.2 | 1.2 |
| Civilian Trauma | −15.3 | −14.0 | −4.2 | −0.5 |

As shown in Table A, in the clinical study the total population of patients achieved an average reduction of 11.8 points in the CAPS-5 scale, an average reduction of 9.6 points in the PCL-5 scale, and an average reduction of 2.9 points in the PHQ-9 scale, after 8 weeks of NF treatment, compared to baseline measurements.

As shown in Table A, in the clinical study a sub-population of female patients achieved an average reduction of 15.3 points in the CAPS-5 scale, and average reduction of 14.6 points in the PCL-5 scale, and an average reduction of 4.6 points in the PHQ-9 scale, after 8 weeks of NF treatment, compared to baseline measurements.

As shown in Table A, in the clinical study a sub-population of male patients achieved an average reduction of 8.6 points in the CAPS-5 scale, an average reduction of 4.8 points in the PCL-5 scale, and an average reduction of 1.3 points in the PHQ-9 scale, after 8 weeks of NF treatment, compared to baseline measurements.

As shown in Table A, in the clinical study a sub-population of patients diagnosed with PTSD originating from non-civilian trauma achieved an average reduction of 7.4 points in the CAPS-5 scale, an average reduction of 3.9 points in the PCL-5 scale, and an average reduction of 1.2 points in the PHQ-9 scale, after 8 weeks of NF treatment, compared to baseline measurements.

As shown in Table A, in the clinical study a sub-population of patients diagnosed with PTSD originating from a civilian trauma (non-non-civilian trauma) achieved an average reduction of 15.3 points in the CAPS-5 scale, an average reduction of 14 points in the PCL-5 scale, and an average reduction of 4.2 points in the PHQ-9 scale, after 8 weeks of NF treatment, compared to baseline measurements.

Table B below summarizes changes in scores of different scales at the 3 month follow up assessment, in the total population and in different sub-populations (compared to baseline scores):

TABLE B

| Change from Baseline: | CAPS-5 | | | PCL-5 | | | PHQ-9 | | | ERQ | | |
|---|---|---|---|---|---|---|---|---|---|---|---|---|
| Month 3 Follow up | N | Mean | SD | N | Mean | SD | N | Mean | SD | N | Mean | SD |
| Total Population | | −13.5 | | | −12.9 | | | −4.0 | | | 1.4 | |
| Females | | −17.6 | | | −16.6 | | | −5.3 | | | 2.4 | |
| Males | | −9.6 | | | −9.3 | | | −2.7 | | | 0.5 | |
| Non-civilian Trauma | | −7.6 | | | −7.2 | | | −2.7 | | | 1.3 | |
| Civilian Trauma | | −17.9 | | | −17.1 | | | −4.9 | | | 1.5 | |

As shown in Table B, in the clinical study the total population of patients achieved an average reduction of 13.5 points in the CAPS-5 scale, an average reduction of 12.9 points in the PCL-5 scale, and an average reduction of 4 points in the PHQ-9 scale, 3 months after the completion of the NF treatment (3 months follow up), compared to baseline measurements.

As shown in Table B, in the clinical study a sub-population of female patients achieved an average reduction of 17.6 points in the CAPS-5 scale, an average reduction of 16.6 points in the PCL-5 scale, and an average reduction of 5.3 points in the PHQ-9 scale, 3 months after the completion of the NF treatment (3 months follow up), compared to baseline measurements.

As shown in Table B, in the clinical study a sub-population of male patients achieved an average reduction of 9.6 points in the CAPS-5 scale, an average reduction of 9.3 points in the PCL-5 scale, and an average reduction of 2.7 points in the PHQ-9 scale, 3 months after the completion of the NF treatment (3 months follow up), compared to baseline measurements.

As shown in Table B, in the clinical study a sub-population of patients diagnosed with PTSD originating from non-civilian trauma achieved an average reduction of 7.6 points in the CAPS-5 scale, an average reduction of 7.2 points in the PCL-5 scale, and an average reduction of 2.7 points in the PHQ-9 scale, 3 months after the completion of the NF treatment (3 months follow up), compared to baseline measurements.

As shown in Table B, in the clinical study a sub-population of patients diagnosed with PTSD originating from a civilian trauma achieved an average reduction of 17.9 points in the CAPS-5 scale, an average reduction of 17.1 points in the PCL-5 scale, and an average reduction of 4.9 points in the PHQ-9 scale, 3 months after the completion of the NF treatment (3 months follow up), compared to baseline measurements.

FIG. 6A shows a boxplot depicting the distribution of the CAPS-5 score of the population in a study at 3 time points 1. Baseline, 2. After 8 weeks of treatment, 3. Three months following the last NF session (i.e. 5 months from start of treatment).

As can be seen, the average CAPS-5 score dropped from 40 to 26.8 points, after 8 weeks of NF training.

FIG. 6B shows a box plot depicting the distribution of the cluster B score of the CAPS-5 (Re-experiencing) of the population in the study at 3 time points and the reduction over time.

FIG. 6C shows a box plot depicting the distribution of the cluster C score of the CAPS-5 (Avoidance) of the population in the study at 3 time points and the reduction over time.

FIG. 6D shows a box plot depicting the distribution of the cluster D score of the CAPS-5 (negative alterations in mood) of the population in a study at 3 time points and the reduction over time.

FIG. 6E shows a box plot depicting the distribution of the cluster E score of the CAPS-5 (Alterations in arousal and reactivity) of the population in THE study at 3 time points and the reduction over time.

FIG. 6F shows a box plot depicting the distribution of the cluster G score of the CAPS-5 (Disturbance causing impairment) of the population in a study at 3 time points and the reduction over time.

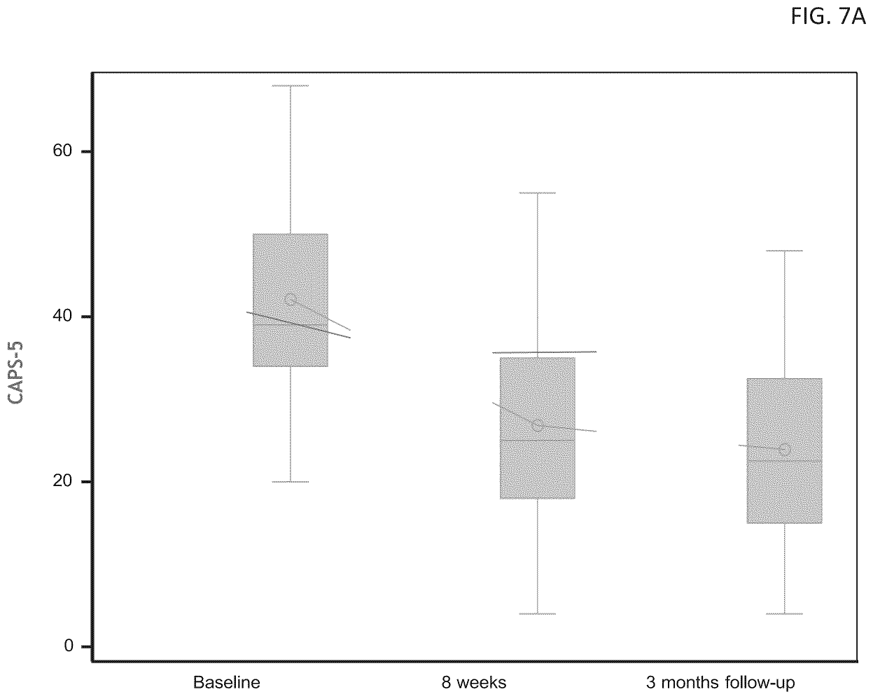

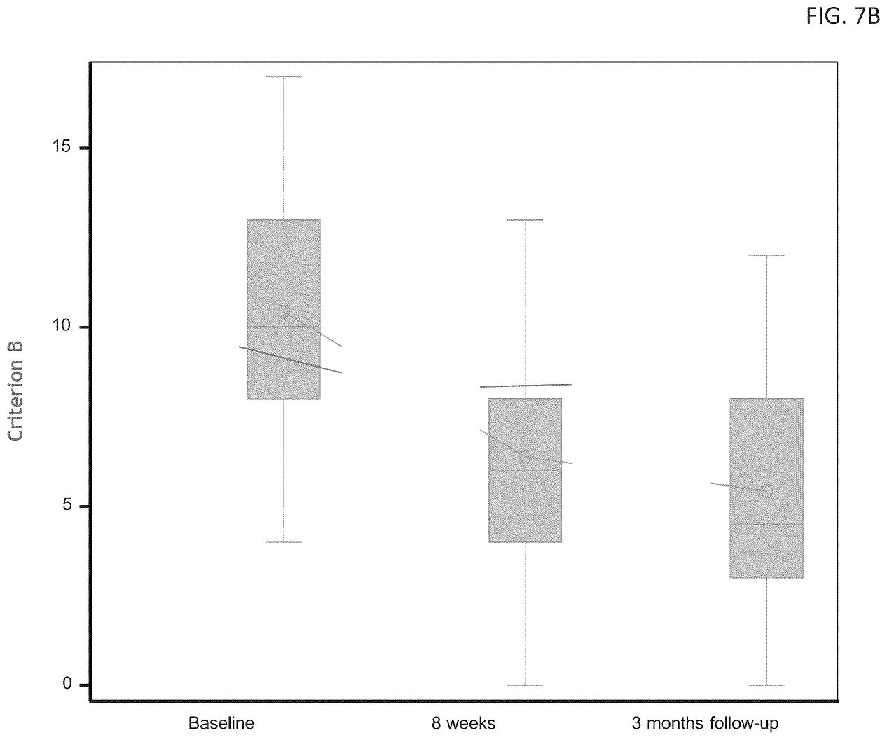

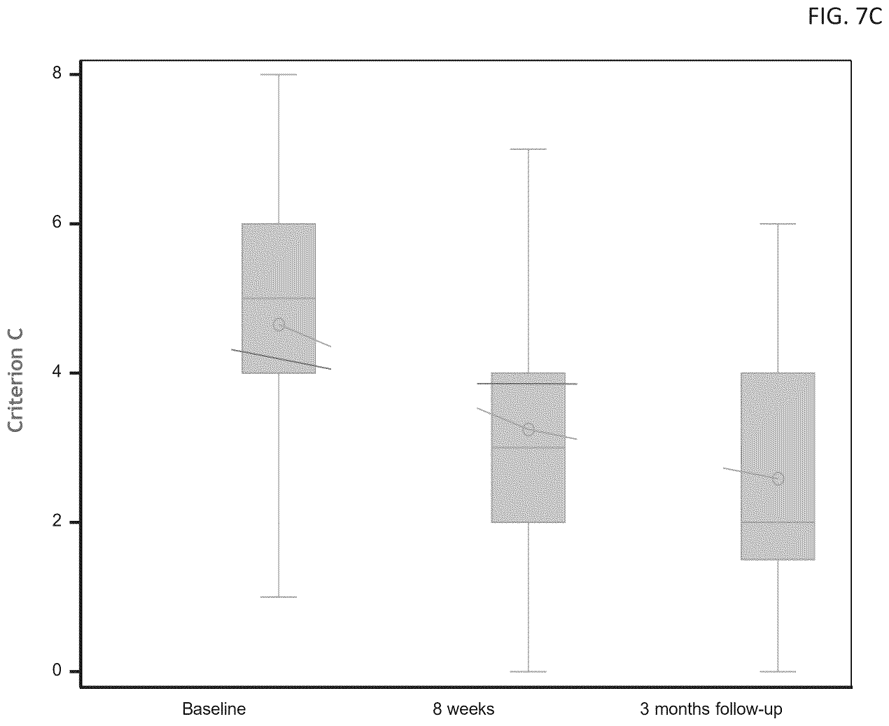

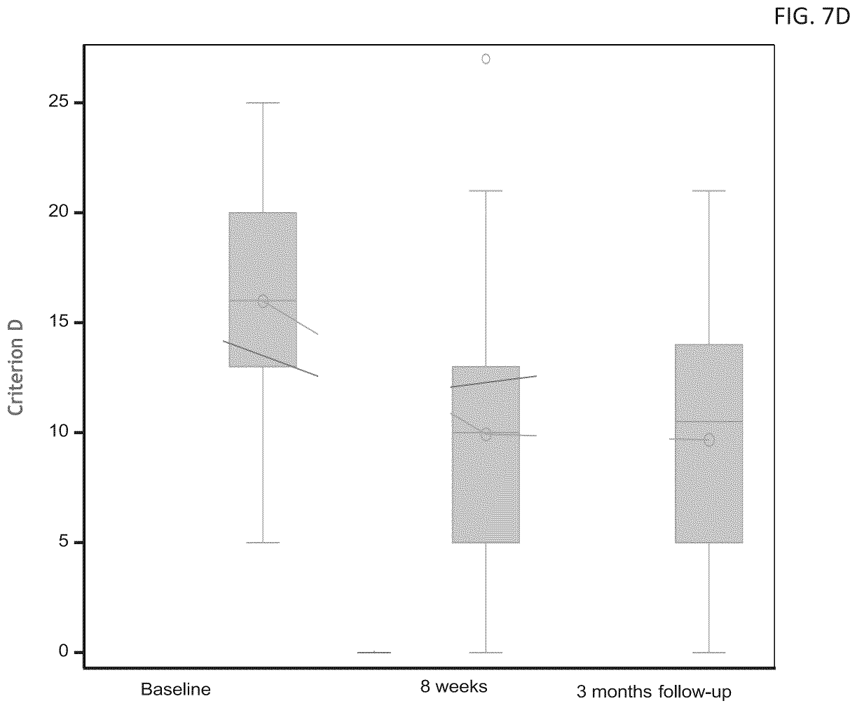

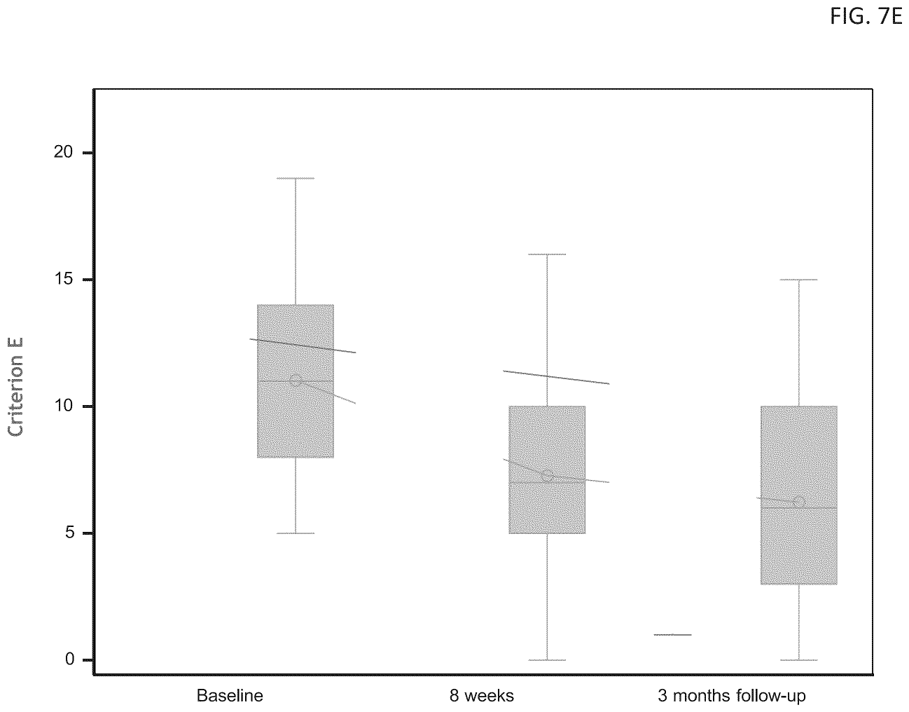

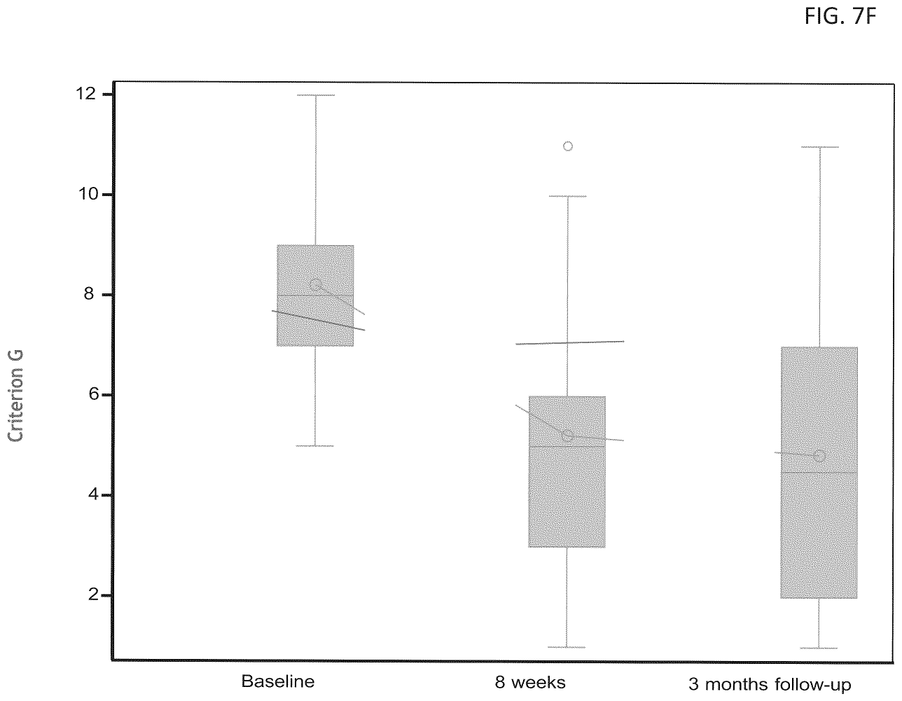

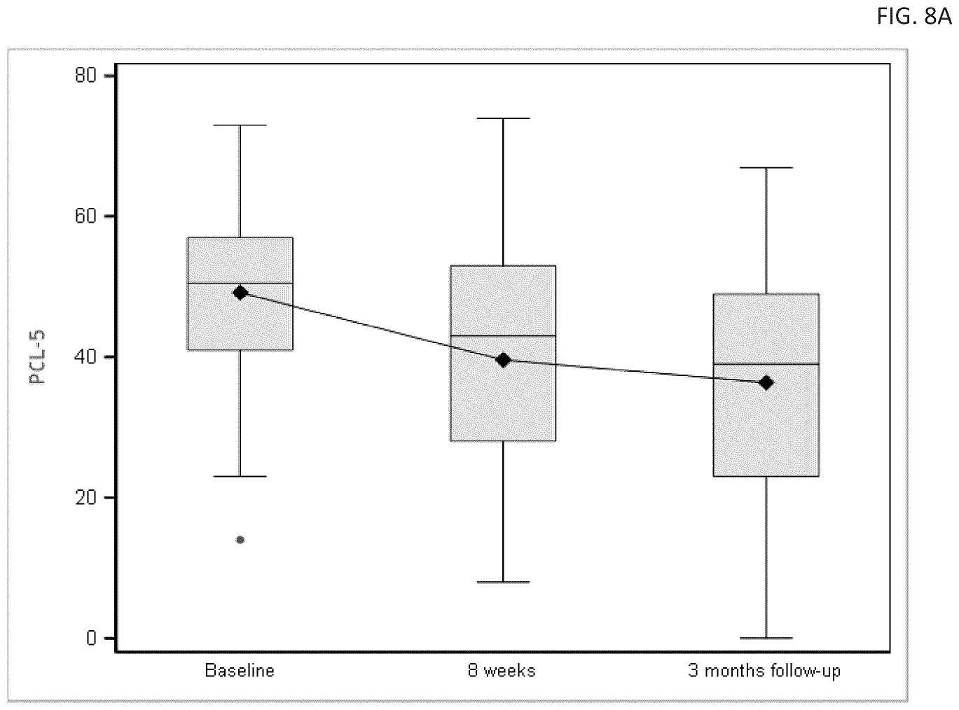

FIG. 7A shows a box plot depicting the CAPS-5 score distribution of the civilian trauma population in the study at 3 time points and the reduction over time.

FIG. 7B shows a box plot depicting the distribution of the cluster B score of the CAPS-5 (Re-experiencing) of the civilian trauma population in the study at 3 time points and the reduction over time.

FIG. 7C shows a box plot depicting the distribution of the cluster C score of the CAPS-5 (Avoidance) of the civilian trauma population in a study at 3 time points and the reduction over time.

FIG. 7D shows a box plot depicting the distribution of the cluster D score of the CAPS-5 (negative alterations in mood) of the civilian trauma population in the study at 3 time points and the reduction over time.

FIG. 7E shows a box plot depicting the distribution of the cluster E score of the CAPS-5 (Alterations in arousal and reactivity) of civilian trauma populations in a study at 3 time points and the reduction over time.

FIG. 7F shows a box plot depicting the distribution of the cluster G score of the CAPS-5 (Disturbance causing impairment) of the civilian trauma population in the study at 3 time points and the reduction over time

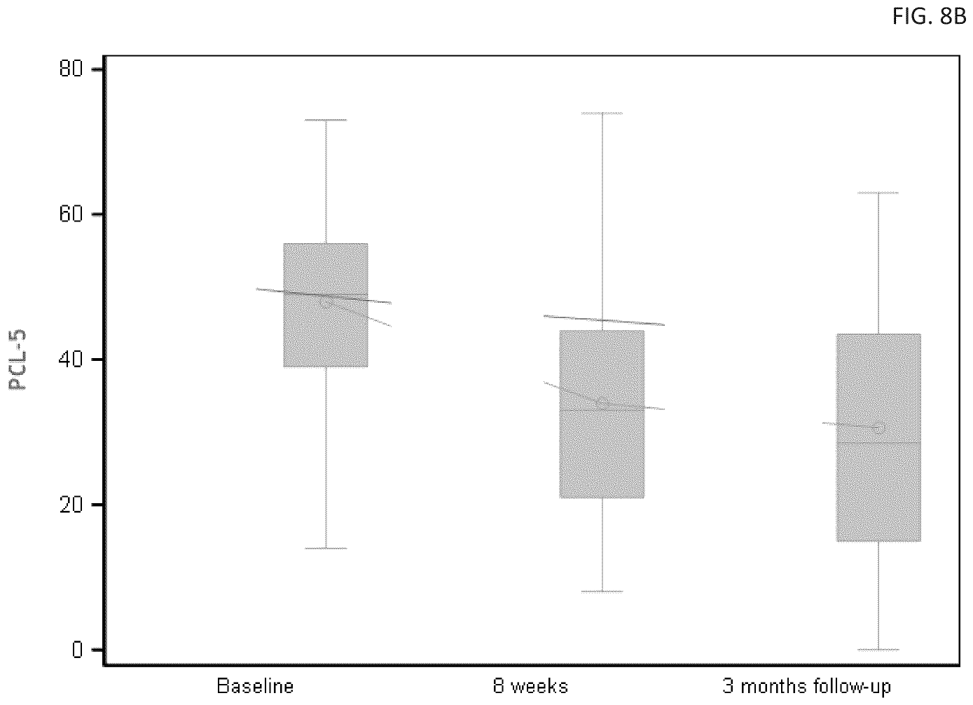

FIG. 8A shows a boxplot depicting the distribution of the PCL-5 score of the population in a study at 3 time points 1. Baseline, 2. After 8 weeks of treatment, 3. Three months following the last NF session (i.e. 5 months from start of treatment).

FIG. 8B shows a box plot depicting the PCL-5 score distribution of the civilian trauma population in a study at 3 time points and the reduction over time.

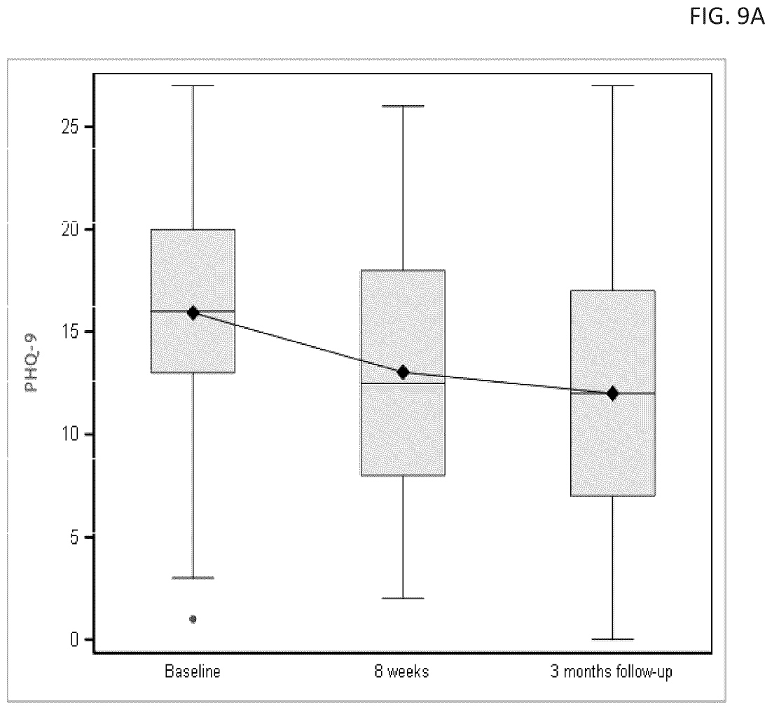

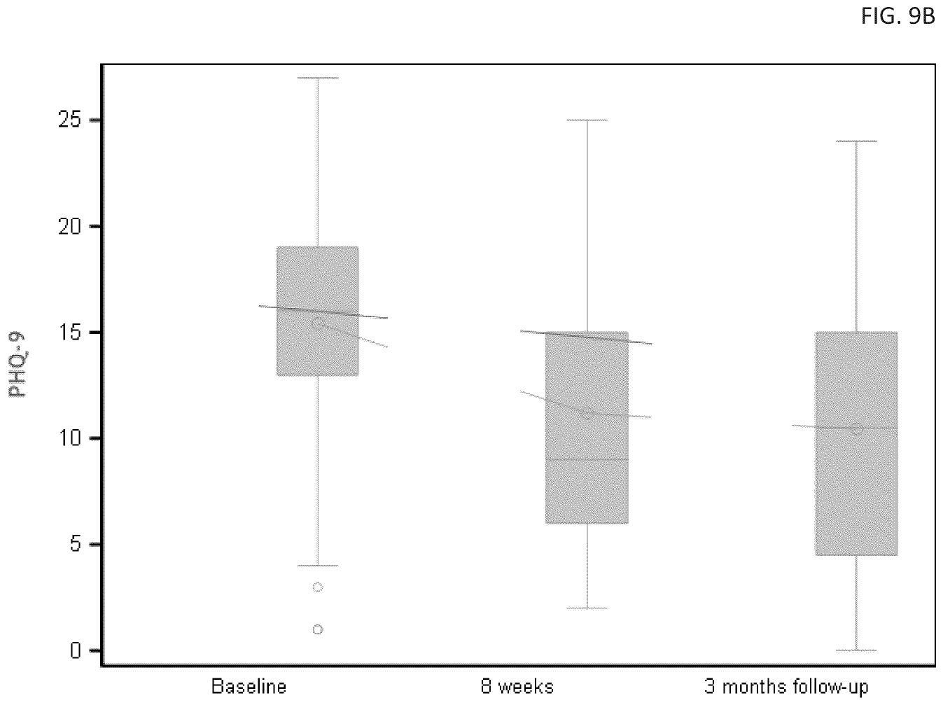

FIG. 9A shows a boxplot depicting the distribution of the PHQ-9 score of the population in a study at 3 time points 1. Baseline, 2. After 8 weeks of treatment, 3. Three months following the last NF session (i.e. 5 months from start of treatment)

FIG. 9B shows a box plot depicting the PHQ-9 score distribution of the civilian trauma population in a study at 3 time points and the reduction over time.

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1-50. (canceled)

51. A method for treating post-traumatic stress disorder (PTSD), comprising:
classifying PTSD patients according to gender and/or trauma origin;
selecting a specific sub-population of patients according to said classifying;
delivering a neurofeedback (NF) treatment to said specific sub-population of patients;
achieving a reduction of at least 6 points in average in a Clinician-Administered PTSD Scale for DSM-5 (CAPS-5) scale in said selected specific sub-population, after a time period between 8 weeks and 12 weeks of said delivering, or up to 3 months from said delivering.

52. A method according to claim 51, wherein said achieving comprises achieving a reduction of at least 4 points in average in a PTSD Checklist for DSM-5 (PCL-5) scale in said selected specific sub-population, after a time period of at least 8 weeks of said delivering.

53. A method according to claim 51, wherein said achieving comprises achieving a reduction of between 8 and 17 points in average in a PCL-5 scale in said selected specific sub-population, up to 3 months from said delivering.

54. A method according to claim 51, wherein said achieving comprises achieving a reduction of between 2 and 5 points in average in a Patient Health Questionnaire-9 (PHQ-9) scale in said selected specific sub-population, up to 3 months from said delivering.

55. A method according to claim 51, wherein said selecting comprises selecting a specific sub-population of females diagnosed with PTSD, and wherein said achieving comprises achieving a reduction of at least 10 points in average, in a CAPS-5 scale in said selected specific sub-population of females, after a time period of at least 8 weeks of said delivering.

56. A method according to claim 55, wherein said achieving comprises achieving a reduction of at least 9 points in average, in a PCL-5 scale in said selected specific sub-population of females, after a time period of at least 8 weeks of said delivering.

57. A method according to claim 55, wherein said achieving comprises achieving a reduction of at least 2 points in average, in a PHQ-9 scale in said selected specific sub-population of females, after a time period of at least 8 weeks of said delivering.

58. A method according to claim 51, wherein said selecting comprises selecting a specific sub-population of males diagnosed with PTSD, and wherein said achieving comprises achieving a reduction of at least 6 points in average, in a CAPS-5 scale in said selected specific sub-population of males, after a time period of at least 8 weeks of said delivering.

59. A method according to claim 58, wherein said achieving comprises achieving a reduction of at least 4 points in average, in a PCL-5 scale in said selected specific sub-population of males, after a time period of at least 8 weeks of said delivering.

60. A method according to claim 58, wherein said achieving comprises achieving a reduction of at least 1 point in average, in a PHQ-9 scale in said selected specific sub-population of males, after a time period of at least 8 weeks of said delivering.

61. A method according to claim 51, wherein said selecting comprises selecting a specific sub-population of patients diagnosed with PTSD, in which said PTSD originates from non-civilian trauma.

62. A method according to claim 61, wherein said achieving comprises achieving a reduction of at least 3 points in average, in a PCL-5 scale in said selected specific sub-population of PTSD patients in which said PTSD originates from non-civilian trauma, after a time period of at least 8 weeks of said delivering.

63. A method according to claim 61, wherein said achieving comprises achieving a reduction of at least 1 point in average, in a PHQ-9 scale in said selected specific sub-population of PTSD patients in which said PTSD originates from non-civilian trauma, after a time period of at least 8 weeks of said delivering.

64. A method according to claim 51, wherein said selecting comprises selecting a specific sub-population of patients diagnosed with PTSD, in which said PTSD originates from a civilian trauma, and wherein said achieving comprises achieving a reduction of at least 10 points in average, in a CAPS-5 scale in said selected specific sub-population of patients in which said PTSD originates from a civilian trauma, after a time period of at least 8 weeks of said delivering.

65. A method according to claim 64, wherein said achieving comprises achieving a reduction of at least 10 points in average, in a PCL-5 scale in said selected specific sub-population of PTSD patients in which said PTSD originates from a civilian trauma, after a time period of at least 8 weeks of said delivering.

66. A method according to claim 64, wherein said achieving comprises achieving a reduction of at least 3 points in average, in a PHQ-9 scale in said selected specific sub-population of PTSD patients in which said PTSD originates from a civilian trauma, after a time period of at least 8 weeks of said delivering.

67. A method according to claim 51, wherein said delivering comprises training said selected sub-population using said NF treatment to modulate activation of the Amygdala or activation of at least one related brain region or at least one biomarker thereof.

68. A system for delivering of a neurofeedback treatment for treating PTSD, comprising:
at least one user interface, configured to deliver at least one indication;
a control unit, comprising:
a memory, wherein said memory stores at least one protocol of a neurofeedback (NF) treatment or indications thereof, and information about classification of at least one PTSD patient according to gender and/or according to trauma origin;
and a control circuitry, wherein said control circuitry is configured to deliver said NF treatment to said PTSD patient based on said at least one protocol or indications thereof stored in said memory, and to signal said at least user interface to generate an indication to perform an assessment of results of said NF treatment within a time period between 8 weeks and 12 weeks from initiating the delivery of said NF treatment.

69. A system according to claim 68, wherein said control unit is configured to process the assessment results and generate a human detectable indication based on the processed assessment results.

70. A dynamic audio-visual interface delivered during a PTSD treatment to a PTSD patient classified according to gender and/or trauma origin, wherein said dynamic audio-visual interface is configured to be modified during said treatment to achieve a reduction of at least 6 points in a Clinician-Administered PTSD Scale for DSM-5 (CAPS-5) in said PTSD patient after a time period of between 8 weeks and 12 weeks of said treatment, or up to 3 months from completion of said treatment.
